(19) United States
(12) Patent Application Publication
(10) Pub. No.: US 2020/0078840 A1
(43) Pub. Date: Mar. 12, 2020

(54) ECOLOGICAL POLLUTION TREATMENT SYSTEM FOR LIVESTOCK AND POULTRY FARMS BASED ON COMBINATION OF PLANTING AND BREEDING

(71) Applicants: Shen WANG, CHANGSHA (CN); HUNAN SAKAL ENVIRONMENTAL SCIENCE AND TECHNOLOGY CO., LTD., CHANGSHA (CN)

(72) Inventor: Shen WANG, CHANGSHA (CN)

(73) Assignee: HUNAN SAKAL ENVIRONMENTAL SCIENCE AND TECHNOLOGY CO., LTD.

(21) Appl. No.: 16/529,304

(22) Filed: Aug. 1, 2019

Related U.S. Application Data
(63) Continuation of application No. PCT/CN2017/106630, filed on Oct. 18, 2017.

(30) Foreign Application Priority Data
Oct. 12, 2017 (CN) 201710949791.6

(51) Int. Cl. B09B 3/00; B09B 5/00; C05F 17/00; C05F 3/06; A23K 10/20

(52) U.S. Cl. CPC B09B 3/0091; B09B 5/00; A01K 67/0332; C05F 3/06; A23K 10/20; C05F 17/0009

(57) ABSTRACT

An ecological pollution treatment system for livestock and poultry farms based on combination of planting and breeding, including a source separation water-saving enclosure, a pollution treatment factory, a feed factory and a supporting planting land. The pollution treatment factory and the feed factory are managed by professional teams for specialized disposal of livestock and poultry manure. The pollution treatment factory is composed of a high-temperature aerobic solid fermentation system, a medium-temperature anaerobic liquid fermentation system, a cracking and propagation system, a heating and heat balancing system, a waste gas treatment system and a detection and control system. Sensors of the detection and control system are arranged in the above systems. The present invention can carry out comprehensive treatment on feces, urine and other wastes and waste gases generated during breeding in large-scale pig farms for resource utilization, and is of great significance to rural environmental protection.

### CROSS-REFERENCE TO RELATED APPLICATIONS

This application is a continuation of International Patent Application No. PCT/CN2017/106630 with a filing date of Oct. 18, 2017, designating the United States, now pending, and further claims priority to Chinese Patent Application No. 201710949791.6 with a filing date of Oct. 12, 2017. The content of the aforementioned applications, including any intervening amendments thereto, are incorporated herein by reference.

### TECHNICAL FIELD

The present invention belongs to the technical field of environmental protection, and particularly relates to an ecological pollution treatment system for livestock and poultry farms based on combination of planting and breeding.

### BACKGROUD OF THE PRESENT INVENTION

In recent years, rapid intensive and large-scale development of the breeding industry has provided abundant and high-quality livestock and poultry products for the market, and has also brought a large amount of pollutants such as feces and urine, and sick and dead livestock and poultry, which will cause serious pollution to rural environments without timely and effective disposal. Recently, a series of standards and policies, e.g., the Technical Specifications for Application of Biogas Fertilizers (NT/T2065-2011), have been issued in China while vigorously supporting and promoting large-scale and intensive livestock and poultry fanning.

Aerobic fermentation (composting) can degrade organic matters by microorganisms to realize reducing, harmless and resourceful treatment of organic wastes. At present, compared with traditional site composting, the aerobic fermentation mode of a reactor has advantages of high fermentation efficiency, complete harmlessness, high product quality and stability, small occupied site area, no influence from environmental factors and difficulty to produce secondary pollution, and is increasingly welcomed by customers. At the beginning of aerobic fermentation of the reactor, exogenous heating materials are often adopted to establish suitable conditions for efficient propagation of thermophilic microorganisms and decomposition of the organic matters. However, problems such as slow heating, long heating time and high heating energy consumption are often encountered in actual production.

At present, according to the Ministry of Agriculture standard NY/T2374-2013 Technical Specifications for Post-treatment of Biogas Shiny and Biogas Residues from Biogas Projects, to achieve harmlessness of the biogas slurry requires lasting the anaerobic fermentation under normal temperature conditions for more than 30 days and requires lasting the anaerobic fermentation longer in winter due to low environment temperature; thereby requiring that biogas digesters supported by the farms are large enough and infrastructure costs of the biogas digesters are high. Research results show that biogas production shows positive correlation with temperature in biogas digesters within a certain temperature range; and the higher the temperature is, the higher the biogas production rate is. Therefore, appropriate increasing of the temperature in the biogas digesters is important to shorten harmlessness time of the biogas slurry and increase the biogas production rate.

It is estimated that the annual mortality rate of adult pigs is 2%-3%, the mortality rate of middle pigs is 7%-8%, and the mortality rate of suckling pigs is up to 10%. In the event of an epidemic, the proportion will be even greater. A problem of treating this part of sick and dead pigs carrying harmful germs needs to be solved urgently. At present, methods for harmless treatment of sick and dead livestock and poultry mainly include incineration, landfilling, high-temperature composting and chemical production methods. The water content of livestock and poultry exceeds 70%, so incineration is relatively hard to be realized and the energy consumption is high. Serious secondary pollution may be easily produced due to high water content and insufficient combustion. At present, the landfilling mode is commonly applied. Animal carcasses are landfilled by digging in the wild or in the place where the epidemic occurred. The landfilling costs are high, but the subsidies for the sick and dead livestock and poultry are much lower than the landfilling costs. Most of workers of the farms fail to do sanitary landfilling due to lack of health and safety knowledge, thereby causing serious secondary pollution. The high-temperature composting method has disadvantages that mechanical loss, energy consumption and equipment cleaning in crushing of the sick and dead livestock and poultry and other pretreatment processes will increase treatment costs; operators will be prone to cross infection in processes of crushing of the sick and dead livestock and poultry, equipment cleaning and replacement of wearing part, thereby increasing a risk of epidemic spread; the composting method is large in occupied area, long in time and easy to be affected by climatic conditions; and the sick and dead livestock and poultry carry a large number of pathogenic microorganisms, which are hard to be completely killed by traditional fermentation modes. The chemical production method is a method for realizing complete harmlessness of the sick and dead livestock and poultry by high temperature and high pressure in a closed container. A large amount of condensed water and cracking liquid are generated during treatment with the traditional methods. The condensed water still needs to be discharged after being environmentally treated to reach a standard. The cracking liquid is generally dried to form powder. The chemical production method has disadvantages that the energy consumption is high in the drying process, a large amount of condensed water is generated, and the treatment costs are greatly increased.

CN105689364A discloses a method for harmless treatment of sick and dead pigs, including: disintegrating sick and dead pig bodies at first; directly sterilizing, drying or carbonizing with hot gas at 120-320° C.; pressing the sterilized and dried meat, hair and bone substances; flowing oil obtained during pressing into a collecting tank; and preparing the remaining meat, hair and bone substances after pressing into animal feed or carbonizing and then landfilling. The method has the following disadvantages: (1) the process of crushing the sick and dead pigs increases the treatment costs, is prone to cross infection, and increases the risk of epidemic spread; (2) the process of drying or carbonizing has high energy consumption, long treatment time and high cost, also generates a lot of waste water and waste gas, has high treatment costs and is prone to secondary pollution; and (3) the sick and dead pigs are rich in organic matters such as protein and fat, major, medium and trace elements as well as inorganic salts, so the landfilling wastes resources and has a low resource utilization rate.

Commercial feed is one of main costs of livestock and poultry farming. The cost is high if only the commercial feed is used for feeding. The livestock and poultry may be overfat, which may affect growth and decline meat quality if they are fed with the commercial feed only in some stages of breeding.

### SUMMARY OF PRESENT INVENTION

A technical problem to be solved by the present invention is to provide an ecological pollution treatment system and method for livestock and poultry farms based on combination of planting and breeding, for ecologically treating pollution of the livestock and poultry farms and achieving purposes of zero discharge, zero pollution and resource utilization, in order to solve problems existing in the background.

The present invention adopts a technical solution as follows. The ecological pollution treatment system for livestock and poultry farms based on combination of planting and breeding includes a source separation water-saving enclosure, a pollution treatment factory, a feed factory and a supporting planting land. The pollution treatment factory and the feed factory are managed by professional teams for specialized disposal of livestock and poultry feces. The pollution treatment factory is specialized in transforming and preparing livestock and poultry manure into fertilizers needed to improve soil by utilizing a supporting pollution treatment workshop at source. The feed factory is specialized in transforming and preparing the livestock and poultry manure into animal-derived feed and plant-derived feed by utilizing the supporting planting land and a feed production workshop at source. The pollution treatment factory is composed of a high-temperature aerobic solid fermentation system, a medium-temperature anaerobic liquid fermentation system, a cracking and propagation system, a heating and heat balancing system, a waste gas treatment system and a detection and control system. The source separation water-saving enclosure is a water-saving enclosure configured to separate rainwater and sewage, drinking water and sewage, discharge the rainwater and the remaining drinking water to outdoor ditches instead of mixing into the manure, prevent the rainwater and the remaining drinking water from mixing into the manure at source of the enclosure by means of water-saving enclosure flushing and mechanical manure scraping or manual dry manure cleaning to minimize the manure, and flush the enclosure with high-pressure spray nozzles or even high-pressure air when slaughtering or transferring the livestock and poultry. The manure cleaned by mechanical manure scraping or manual dry manure cleaning is piled up in a dry manure shed. Then, the manure is conveyed to the high-temperature aerobic solid fermentation system and the feed factory by the manure shed, respectively. Frass generated in the feed factory is conveyed to the high-temperature aerobic solid fermentation system, while manure liquid and enclosure flushing water are conveyed to an adjusting tank. The adjusting tank is connected with a feed port of the medium-temperature anaerobic liquid fermentation system and a liquid inlet of the cracking and propagation system. An exhaust port of fermentation odor generated by the high-temperature aerobic solid fermentation system and an exhaust port of fermentation odor generated by the cracking and propagation system are connected with the waste gas treatment system by exhaust pipes. The heating and heat balancing system is respectively connected with a heating jacket or coil of the high-temperature aerobic solid fermentation system, heating coils of the medium-temperature anaerobic liquid fermentation system as well as a water jacket and a coil of the cracking and propagation system by pipes. Sensors of the detection and control system are arranged in the above systems, to set, detect and control key parameters.

In the above technical solution, the feed factory is managed by the professional teams for specialized disposal of livestock manure and sewage, and is specialized in blending the livestock and poultry manure as well as cracking liquid of the dead livestock and poultry and placentas to prepare livestock and poultry manure for feeding insects to produce insects and ova and serving as the animal-derived feed while producing the plant-derived feed by using the livestock and poultry manure to plant grains and pastures on the supporting planting land at source. The planted grains and pastures include, but not limited to one or more of Pennisetum hydridum, Pennisetum purpureum, Alfalfa, Alternanthera, Lolium perenne, Chinese pennisetum, Broussonetia papyrifera, corns and soybeans. The bred insects include, but not limited to one or more of black soldier fly and earthworm. The feed factory is specialized in adding appropriate grains, trace elements and other ingredients into the obtained plant-derived feed and animal-derived feed to produce complete nutrition feed for breeding the livestock and poultry.

The high-temperature aerobic solid fermentation system includes M high-temperature aerobic solid fermentation reactors, wherein M is a positive integer. The dry manure shed is respectively communicated with the feed port of each high-temperature aerobic solid fermentation reactor by the conveying device. An aging chamber communicated with each high-temperature aerobic solid fermentation reactor is arranged at a discharge port of each high-temperature aerobic solid fermentation reactor.

The medium-temperature anaerobic liquid fermentation system includes the adjusting tank, N medium-temperature anaerobic liquid fermentation reactors, a liquid outlet tank, a sludge pump and a liquid storage tank communicated in sequence, wherein N is a positive integer.

Further, the medium-temperature anaerobic liquid fermentation reactors are soft anaerobic fermentation bags. A liquid outlet of the adjusting tank is connected with the feed port of a first soft anaerobic fermentation bag through a pipe; and the discharge port of the soft anaerobic fermentation bag is connected with the feed port of a second soft anaerobic fermentation bag through the pipe, and so on, until the discharge port is connected to the feed port of an Nth soft anaerobic fermentation bag, and the discharge port thereof is connected with the liquid outlet tank through the pipe. Then, the liquid outlet tank is connected with the liquid storage tank by the sludge pump.

Further, the medium-temperature anaerobic liquid fermentation reactors include reaction tanks having rectangular bottoms. Each reaction tank is arranged on an inclined surface with an inclination angle of 0.3-1% in a length direction of the reaction tank. A drainage ditch is formed around the inclined surface. The drainage ditch is connected to a water collecting well at the lower ground through the pipe. Water collected at the bottoms of the reaction tanks is converged to the water collecting well through the drainage ditch for drainage. Thermal insulation layers are arranged around the bottoms of the reaction tanks and the inclined surface at the bottoms of the tanks. The thermal insulation layers are made of thermal insulation materials. A heat radiation plate is arranged on the surface of the thermal insulation layer at the bottoms of the tanks. The heating coils are uniformly fixed on the heat radiation plate. The heating coils are covered with the soft anaerobic fermentation bags. The feed port is formed in a higher side of each soft anaerobic fermentation bag, and the discharge port is formed in a lower side of the same in the length direction. In order to prevent liquid surfaces in biogas bags from crusting, a polarizer is arranged in the middle of each rectangular reactor in the length direction; biogas pipes are arranged at the tops of the soft anaerobic fermentation bags; pressure sensors are arranged on the pipes; and the soft anaerobic fermentation bags are further covered with the thermal insulation layers and waterproof covers.

Further, in the medium-temperature anaerobic liquid fermentation reactors connected in series with each other, the height of the feed port of a latter medium-temperature anaerobic liquid fermentation reactor is more than 0.2 in lower than that of the discharge port of a previous medium-temperature anaerobic liquid fermentation reactor to prevent sediments in the subsequent reactor from flowing back to the previous reactor. For each medium-temperature anaerobic liquid fermentation reactor, the tank bottom at a feed port side is higher than that at a discharge side; and a range of inclination is 0.3-1% to reduce the number of sludge cleaning times of the medium-temperature anaerobic liquid fermentation reactors.

Further, the cracking and propagation system is composed of Y cracking and propagation reactors (Y≥2), X cracking reactors (X≥1), Z propagation reactors (Z≥1), steam generators, aeration fans, air filters, electromagnetic valves and connecting pipes. Liquid in the adjusting tank is transferred to the cracking and propagation. reactors and the cracking reactors by transfer pumps. Discharge pipes of the cracking reactors are connected to the propagation reactors. Discharge pipes of the cracking and propagation reactors and the propagation reactors are respectively connected to the liquid storage tank through pipes.

Further, each cracking and propagation reactor includes a support, a tank body, a sealing cover and a conveying device. Each tank body is fixed on a base. One side of each tank body is sealed by the sealing cover. A cover sealing door is mounted at the other side of each tank body. Each cover sealing door is hinged to the tank body. The cover sealing door is locked and sealed with a plurality of locking bolts when closed, so that a closed cracking and propagation space is formed among the sealing cover, the tank body and the cover sealing door. The tank body is horizontally arranged. Guide rails parallel to an axis of the tank body and radially fixed along the tank body are arranged in the tank body for bearing and conveying net cages carrying the sick and dead pigs and the placentas. Aeration pipes and a plurality of aeration heads are mounted at lower parts of the guide rails. One end of each aeration pipe is sealed, while the other end extends to the outside of the tank body by the sealing cover and is sequentially communicated with air outlets of the electromagnetic valve, a one-way valve, the air filters and the aeration fan and steam, outlets of the electromagnetic valve, the one-way valve and a steam generator. An exhaust pipe and a feed pipe are arranged on an upper side of each sealing cover. A pressure sensor and a safety valve are further mounted on the upper side of the sealing cover. A temperature sensor and a discharge pipe are mounted on a lower side of the sealing cover. A water jacket is mounted outside each horizontally arranged tank body for cooling each cracking tank. A circulating water inlet pipe of the water jacket is arranged at a lower part of the tank body. A circulating water drainage pipe of the water jacket is arranged at an upper part of the tank body. The water jacket is covered with the thermal insulation layer made of the thermal insulation material.

In the above technical solution, in order to timely and harmlessly treat the sick and dead livestock and poultry (especially small-sized livestock and poultry) and the placentas, the racking-reactors are also provided. Each cracking reactor is composed of a support body; a sealing cover and a conveying device. Each tank body is fixed on a base. One side of the tank body is sealed by the sealing cover. A cover sealing door is mounted at the other side of the tank body. Each cover sealing door is hinged to the tank body. The cover-sealing door is locked and sealed with a plurality of lock bolts when closed, so that a closed cracking and propagation space is formed among the sealing cover, the tank body and the cover sealing door. Further, the tank body is horizontally arranged. Guide rails parallel to an axis of the tank body and radially fixed along the tank body are arranged in the tank body for bearing and conveying net cages carrying the sick and dead pigs and the placentas. Aeration pipes and a plurality of aeration heads are mounted at lower parts of the guide rails. The other end of each aeration pipe is sealed, extends to the outside of the tank body by the sealing cover and is sequentially communicated with the steam outlets of the electromagnetic valve, the one-way valve and the steam generator. An exhaust pipe and a feed pipe are arranged at an upper part of the sealing cover. A pressure sensor and a safety valve are further mounted at the upper part of the sealing cover. A temperature sensor and a discharge pipe are Mounted at a lower part of the sealing cover. A water jacket is mounted outside each horizontally arranged tank body for cooling each cracking tank. A circulating water inlet pipe of the water jacket is arranged at the lower part of the tank body. A circulating water drainage pipe of the water jacket is arranged at the upper part of the tank body. The water jacket is covered with the thermal insulation layer made of the thermal insulation material.

Further, each conveying device includes a trolley and a net cage. A guide rail is arranged at the upper part of the trolley. The net cage is placed on the guide rail. The net cage is a rectangular cage composed of a cage body and steel wire meshes. The surrounding and bottom steel wire meshes are welded on the cage body. A movable cage cover is arranged at the top of the cage body. The cage cover is hinged to the cage body. A handle is further arranged outside the cage cover. A plurality of rollers are fixed at the bottom of the cage body. The rollers are in contact with the guide rail.

The direction of the guide rail on the trolley is the same as that of the guide rail in the tank body, and is flush with the guide rail in the tank. When the sick and dead pigs are conveyed, the sick and dead pigs are first placed-in the net cage, and then are conveyed to a specified position in the tank body by the net cage along the guide rails in the trolley and the tank body.

In the above technical solution, in order to treat cracking liquids outputted by the plurality of small cracking reactors, propagation reactors are specially provided. Each propagation reactor is composed of a vertical and closed thermal insulation tank body, a coil and an aeration device. An aeration port, a coil circulating water outlet, a coil circulating water inlet and an exhaust port are formed in the top of the tank body, while a discharge port is formed in the bottom. The coil is fixed in the tank body and is immersed in the cracking liquid. The aeration device is arranged at the bottom of the tank body. The aeration pipe is connected with the aeration port and is sequentially connected with air outlets of the electromagnetic valve, the air filter and the aeration fan. The thermal insulation tank body is covered with the thermal insulation layer made of the thermal insulation material.

Further, the heating and heat balancing system is composed of an atmospheric pressure hot water boiler, E high-temperature thermal insulation water tanks (E≥1), F low-temperature thermal insulation water tanks (F≥1), circulating water pumps, electromagnetic valves and connecting pipes. For regions with abundant solar energy resources, the heating and heat balancing system also includes a solar heating system. The high-temperature water tanks are used to provide water sources for the atmospheric pressure hot water boiler, the solar heating system and the steam generator. Water outlet pipes of the high temperature water tanks are respectively communicated with water inlets of the atmospheric pressure hot water. boiler, the solar heating system and the steam generator. Preferably, the high-temperature thermal insulation water tanks are covered with the thermal insulation layers made of the thermal insulation materials. The water outlets of the atmospheric pressure hot water boiler and the solar heating system are communicated with water inlet pipes of the high-temperature water tanks through the respective pipe. The other water outlet pipe of each high-temperature water tank is connected with the water pump to respectively convey hot water to the high-temperature aerobic solid fermentation reactors, the medium-temperature anaerobic liquid fermentation reactors, the cracking and propagation reactors, the water jackets of the cracking reactors and the coils of the propagation reactors. Return water of each reactor is sent back to each high-temperature water tank through respective water returning pipe. The low-temperature water tanks provide water for the high-temperature water tanks. The low-temperature water tanks are arranged above the high-temperature water tanks and automatically replenish water to the high-temperature water tanks under control of the detection and control system. The other water outlet pipe of each low-temperature water tank is respectively connected with the cracking and propagation reactors and the cracking reactors by pumps. Water is sent back to the low-temperature water tanks by each water jacket through respective water returning pipes to realize circulation. Preferably, the low-temperature thermal insulation water tanks are covered with the thermal insulation layer made of the thermal insulation material.

Energy sources of the steam generators and the atmospheric pressure hot water boilers include electricity, biogas, diesel, biomass fuel, coal and solar energy.

Further, the waste gas treatment system includes an odor treatment system of the high-temperature aerobic solid fermentation system and a waste gas treatment system of the cracking and propagation system. The odor treatment system of the high-temperature aerobic solid fermentation system has the following structural connection; the exhaust ports of the high-temperature aerobic solid fermentation reactors are respectively connected with waste gas inlets of heat exchange condensers through respective exhaust pipe; a waste gas outlet of each heat exchange condenser is respectively connected with an input end of a draught fan through the pipe; air inlets of the heat exchange condensers are communicated with atmosphere; air outlets of the heat exchange condensers are respectively connected with the gas inlets of the high-temperature aerobic solid fermentation reactors through the pipes; an output end of each draught fan is respectively connected to a gas inlet of a biological deodorization filter tower in parallel through the pipe; and an exhaust port of the biological deodorization filter tower is communicated with the atmosphere through a vertical pipe. The waste gas treatment system of the cracking and propagation system has the following structural connection: the aeration ports of the cracking and propagation reactors are respectively connected with the air filters and the aeration fans through the pipes; exhaust pipes of the cracking and propagation reactors, the cracking reactors and the propagation reactors are respectively connected to input ends of the draught fans; the output ends of the draught fans are connected with gas inlets of the biological deodorization filter towers through the pipes; and the exhaust ports of the biological deodorization filter towers are communicated with the atmosphere through the vertical pipes.

In the above technical solution, the supporting planting land area is determined by synthesizing livestock and poultry breeding quantity and assimilative capacity of feed crops.

In the above technical solution, the livestock and poultry include pigs, chickens, ducks and geese, and also include ruminant animals such as cows and sheep.

Based on the same inventive concept, the present invention also provides an ecological pollution treatment method for livestock and poultry farms based on combination of planting and breeding, including:

I. Source separation and water saving of enclosure: rainwater and sewage, drinking water and sewage are separated; the rainwater and the remaining drinking water are discharged to ditches outside the enclosure instead of being mixed into feces and urine; water-saving enclosure flushing and mechanical manure scraping or manual dry manure cleaning are adopted to prevent the rainwater and the remaining drinking water from mixing into the feces and urine at source of the enclosure and minimize the feces and urine; the enclosure is cleaned with high-pressure spray nozzles or even high-pressure air; the feces cleaned by mechanical manure scraping or manual dry manure cleaning is piled up in a dry manure shed; then, the feces is conveyed to a high-temperature aerobic solid fermentation system and an animal-derived feed factory by the manure shed, respectively; and feces and urine and enclosure flushing water are conveyed to an adjusting tank.

II. Feed insect breeding: a water content of the feces is adjusted to an appropriate range at first; then, the feces is used as an insect feed for breeding insects to obtain insects and ova, which are used as an animal-derived feed, are mixed with a plant-derived feed in a certain proportion and then are added with appropriate grains, trace elements and other ingredients to produce Complete nutrition feed for breeding the livestock and poultry; and frass is conveyed to high-temperature aerobic solid fermentation reactors by the conveying device for high-temperature aerobic fermentation to obtain an organic solid fertilizer.

III. High-temperature aerobic fermentation of feces:

(1) Tap water is automatically replenished to low-temperature thermal insulation water tanks; the low-temperature thermal insulation water tanks automatically replenish the water to high-temperature thermal insulation water tanks by utilizing a height difference under control of a detection and control system; circulating pumps of an atmospheric pressure hot water boiler and a solar heating system are started; the water of the high-temperature thermal insulation water tanks is transferred to the atmospheric pressure hot water boiler and the solar heating system by the circulating pumps for heating and then is transferred to the high-temperature thermal insulation water tanks for energy storage; electromagnetic valves at front ends of heating water jackets or coils of the high-temperature aerobic solid fermentation reactors are started; hot water circulating pumps are started; and hot water is conveyed to the heating water jackets or the coils of the high-temperature aerobic solid fermentation reactors by the circulating water pumps and the pipes to heat up materials in the high-temperature aerobic solid fermentation reactors.

(2) The feces, the frass and auxiliary materials separated from the enclosure and thermophilic decomposing bacteria are conveyed to the high-temperature aerobic solid fermentation reactors by conveying equipment; the water content of the materials is controlled at 55-65%; and the detection and control system simultaneously starts driving devices of the high-temperature aerobic solid fermentation reactors while adding the materials, so that feeding and stirring are realized in the reactors.

(3) After completing feeding, the detection and control system controls the high-temperature aerobic solid fermentation reactors to stop stirring for a time T1, stir for a time T2, stop stirring for the time T1, and then stir for the time T2; a cycle of stop-stir-stop-stir-stop-stir is a timed stirring program; and meanwhile, the detection and control system automatically starts a draught fan to supply oxygen to fermented materials in the high-temperature aerobic solid fermentation reactors when the high-temperature aerobic solid fermentation reactors stir at the time T2. <1> The hot water enters the heating, jackets or coils to rise the temperature of the materials in the reactors when the detection and control system detects that the temperature of the materials in the high-temperature aerobic solid fermentation reactors is lower than a set temperature H1 of the materials. <2> The timed stirring program is stopped and changed to a temperature-controlled stirring program when the temperature of the materials in the high-temperature aerobic solid fermentation reactors is greater than or equal to H2; the draught fans are started, and the high-temperature aerobic solid fermentation reactors are driven to stir; the timed stirring program is not started until the temperature of the materials in the high-temperature aerobic solid fermentation reactors is lower than H2; the temperature of the materials in the high-temperature aerobic solid fermentation reactors is maintained in H1-H2; and the timed stirring program and the temperature-controlled stirring program of the high-temperature aerobic solid fermentation reactors are adopted to establish an appropriate fermentation temperature and provide sufficient oxygen for the materials in the high-temperature aerobic solid fermentation reactors and establish an appropriate environment for high-temperature aerobic fermentation of feces solids.

(4) A single fermentation is completed by feeding the materials and fermenting the materials for a time T3; the detection and control system controls the high-temperature solid fermentation reactors to stop, immediately feed a part of materials after discharging the same amount of the materials, and then immediately feed a part of the materials after discharging the same amount of the materials every T3 time; similarly, a discharge machine is started at first when discharging the materials; and meanwhile, the detection and control system controls the high-temperature solid fermentation reactors to stir and guide the discharge.

(5) The materials discharged from the high-temperature aerobic solid fermentation reactor are conveyed to the aging chamber by the conveying device; the materials are regularly turned or aerated in this period so that the materials are cooled and lose water until the materials are completely decomposed to prepare an organic fertilizer.

(6) The detection and control system respectively detects the temperature of the materials in each high-temperature aerobic solid fermentation reactor when the M high-temperature aerobic solid fermentation reactors simultaneously ferment, so that the temperature of the materials in each high-temperature aerobic solid fermentation reactor is maintained in H1-H2.

(7) The detection and control system detects and controls the water temperature in the high-temperature thermal insulation water tank to keep the water temperature constant in H3-H4. <1> The circulating pump and the atmospheric pressure hot water boiler are started to heat up the hot water in the high-temperature thermal insulation water tank when the temperature in the high-temperature thermal insulation water tank is lower than H3; and the circulating pump of the solar heating system is started to heat up the hot water in the high-temperature thermal insulation water tank when the temperature of the hot water in a heat collecting water tank of the solar heating system is greater than H3; and <2> the atmospheric pressure hot water boiler is shut down when the temperature in the high-temperature thermal insulation water tank reaches H4.

IV. Medium-temperature anaerobic fermentation of manure liquid:

(1) A mixture of the manure liquid and the enclosure flushing water is conveyed into the adjusting tank, so that a liquid level of the liquid in the adjusting tank keeps rising; the manure liquid naturally flows into the first soft anaerobic fermentation bag along a connecting pipe due to a height difference when the liquid level is higher than a liquid outlet of the adjusting tank; the detection and control system controls to open the electromagnetic valve at the front end of the heating coil of the first medium-temperature anaerobic liquid fermentation reactor, and the circulating water pump is started, so that the hot water enters the heating coil for circulation to rapidly rise the temperature of the materials in the soft anaerobic fermentation hag to a set temperature, and the materials start to perform a medium-temperature anaerobic fermentation reaction.

(2) The liquid naturally flows into the second soft anaerobic fermentation bag along the connecting pipe due to the height difference when the liquid level of the liquid in the first soft anaerobic fermentation bag gradually rises and is higher than the liquid outlet; the detection and control system controls to open the electromagnetic valve at the front end of the heating coil of the second medium-temperature anaerobic liquid fermentation reactor, so that the hot water enters the heating coil for circulation to rapidly rise the temperature of the materials in the soft anaerobic fermentation bag to a set temperature, and the materials keep performing the medium-temperature anaerobic fermentation reaction.

(3) The manure liquid naturally flows out along the connecting pipe due to the height difference when the liquid level of the liquid in the second soft anaerobic fermentation bag gradually rises and is higher than the liquid outlet, and so on, until the manure liquid directly flows into the liquid outlet tank through the Nth soft anaerobic fermentation bag; the detection and control system controls to open the electromagnetic valve at the front end of the heating coil of the Nth medium-temperature anaerobic liquid fermentation reactor, so that the hot water enters the heating coil for circulation to rapidly rise the temperature of the materials in the Nth soft anaerobic fermentation bag to the set temperature, and the materials keep performing the medium-temperature anaerobic fermentation reaction.

(4) Polarizers in the N medium-temperature anaerobic liquid fermentation reactors are regularly started, respectively, to prevent the liquid in the soft anaerobic fermentation bags from “crusting” and slow down sedimentation of liquid sediments.

(5) The detection and control system respectively controls on-off of the electromagnetic valve in front of the heating coil of each medium-temperature anaerobic liquid fermentation reactor, and respectively controls the temperature of the materials in each soft anaerobic fermentation bag to keep the water-temperature constant within a set temperature range; the manure liquid sequentially flows through N soft anaerobic fermentation bags; and fermentation liquid in the Nth soft anaerobic fermentation bag naturally flows into the liquid outlet tank along the pipe due to the height difference, to prepare biogas slurry.

(5) The detection and control system regularly starts the sludge pump to control the liquid level of the liquid outlet tank according to a set anaerobic fermentation time T and ensures that a residence time of the manure liquid in the medium-temperature anaerobic liquid fermentation reactor reaches T; the biogas slurry in the liquid outlet tank is pumped into the liquid storage tank by the sludge pump after the time for anaerobic fermentation reaches T, so that the liquid level of the liquid in the liquid outlet tank is lowered; and the sludge pump is turned off after the detection and control system detects that the liquid level of the liquid outlet tank reaches a lower limit of the liquid level.

(6) The biogas generated by the N soft anaerobic fermentation bags is conveyed to a biogas pretreatment device through the conveying pipe for treatment, and then is supplied as a combustion fuel to the atmospheric pressure hot water boiler and the steam generator. The atmospheric pressure hot water boiler and the steam generator also use electricity, diesel and biomass as supplementary fuels when the temperature is low in winter.

V. Cracking and propagation of sick and dead livestock and poultry and placentas:

(1) Cracking of sick and dead livestock and poultry and placentas:

<1> A forklift or other transfer equipment is used to place relatively large sick and dead livestock into the net cage; the net cage is pushed into the cracking and propagation reactors by the conveying device; the relatively small sick and dead livestock and poultry and placentas are placed into the net cage; the net cage is pushed into the cracking reactors by the conveying device; the cover sealing door is closed; and the manure liquid in the adjusting tank is conveyed into the cracking and propagation reactors and the cracking reactors by the sludge pumps so that the net cages are semi-immersed in the liquid.

<2> The steam generator is started; the electromagnetic valves on steam inlet Pipes of the cracking and propagation reactors and the cracking reactors are respectively opened; hot steam generated by the steam generators is respectively conveyed into the cracking and propagation reactors and the cracking reactors by the one-way valves so that the temperature and the pressure of the liquid in the cracking and propagation reactors and the cracking reactors are increased; the exhaust valves on the exhaust pipes of the cracking and propagation reactors and the cracking reactors are respectively closed after exhausting cold air in the cracking and propagation reactors and the cracking reactors, so that the temperature and the pressure in the cracking and propagation reactors and the cracking reactors are continuously increased to respectively reach the temperature and the pressure of statutory treatment; the sick and dead livestock and poultry and the placentas start to be cracked at high temperature and high pressure; and the detection and control system detects and controls the temperature and the pressure in the cracking and propagation reactors and the cracking reactors to keep them constant within a statutory range of the temperature and the pressure for a statutory time, so that the sick and dead livestock and poultry are completely harmless, and are disintegrated and dissolved in the liquid.

<3> The electromagnetic valves, on steam inlet pipes of the cracking and propagation reactors and the cracking reactors are respectively closed after high-temperature and high-pressure cracking is completed; the steam generators are turned off after all the reactors complete the high-temperature and high-pressure cracking; the electromagnetic valves on the hot water pipes and the electromagnetic valves on the water inlet pipes of the cracking and propagation reactors and the cracking reactors are respectively opened; the circulating water pumps on the water outlet pipes of the high-temperature thermal insulation water tanks are started; hot water respectively enters water jackets of the cracking and propagation reactors and the cracking reactors for circulation so that, the cracking liquid is cooled and balanced with the temperature of the hot water in the high-temperature thermal insulation water tank; then, the electromagnetic valves on high-temperature hot water pipes are closed; the circulating water pumps on the water outlet pipes of the low-temperature thermal insulation water tanks are started; cold water enters the water jackets of the cracking and propagation reactors and the cracking reactors for circulation so that the cracking liquid is cooled to a set temperature H6; and the circulating water pumps are turned off.

(2) The cracking liquid is used as a culture medium for propagating microorganisms to obtain a microbial culture solution; and the cracking liquid is used as a raw material of the feed insect breeding for breeding feed insects.

(I) A method for propagating the microbial culture solution with the cracking liquid includes:

<1> The electromagnetic valves on discharge pipes of the cracking reactors are opened; the cracking liquid in the cracking reactors is conveyed into the propagation reactors; pre-cultured microbial seed liquid is respectively conveyed into reactors through the feed ports of the cracking and propagation reactors and the propagation reactors; the electromagnetic valves on the aeration pipes of the cracking and propagation reactors and the propagation reactors are respectively opened; the aeration fans are started; fresh air is filtered by the air filters and then is regularly aerated to supply oxygen to the cracking and propagation reactors and the propagation reactors by the one-way valves; meanwhile, the detection and control system detects and controls the temperature in the reactors to keep the temperature in H5-H6; and a detection and control method includes the detection and control system controls the high-temperature thermal insulation water tanks to heat the cracking and propagation reactors and the propagation reactors to H6 when the detection and control system detects that the temperature in the cracking and propagation reactors and the propagation reactors is lower than a lower limit H5, and then is turned off.

<2> A propagation process is completed when the concentration of a culture bacteria solution reaches requirements after the cracking liquid is cultured and propagated for a period of time; the microbial culture solution is discharged into the liquid storage tanks through discharge valves for standing, and then is separated by an oil-water separator to obtain the microbial culture solution and grease; and the grease is used as an industrial raw material.

<3> The detection and control system detects and controls the temperature and the pressure of the materials in each reactor according to different cracking and propagation stages when a plurality of reactors react at the same time, so that the temperature and the pressure in each reactor are maintained within the set ranges.

(II) A method for breeding the feed insects with the cracking liquid includes:

<1> The livestock and poultry feces is added into the cracking liquid and mixed uniformly; a mixture is used as an insect feed for breeding insects to obtain insects and ova, which are used as an animal-derived feed, are mixed with a plant-derived feed in a certain proportion and then are added with appropriate grains, trace elements and other ingredients to produce complete nutrition feed for breeding the livestock and poultry; and <2> the residual mixture of the cracking liquid and the livestock and poultry feces as well as frass are conveyed to the high-temperature aerobic solid fermentation reactors by the conveying device for high-temperature aerobic fermentation to obtain an organic solid fertilizer.

VI. Planting of feed crops:

(1) The microbial culture solution is sprayed to the aged organic solid fertilizer in a certain proportion, and are stirred uniformly to obtain a bio-organic fertilizer; the microbial culture solution is added into the biogas slurry in a certain proportion to obtain a microbial liquid fertilizer; and an appropriate amount of NPK fertilizer is added into the biogas slurry to prepare an organic-inorganic compound liquid fertilizer according to growth demands of the feed crops.

(2) The appropriate amount of bio-organic fertilizer, microbial liquid fertilizer and organic-inorganic compound liquid fertilizer are respectively applied according to the growth demands of the feed crops before and during planting according to the livestock and poultry breeding quantity of the livestock and poultry farms and the supporting planting land with assimilative capacity of the planted feed crops, to obtain feed ingredients such as pastures and corns; the feed ingredients are conveyed to the feed factory and are processed to prepare the plant-derived feed, which is mixed with the animal-derived feed in a certain proportion and then is added with appropriate grains, trace elements and other ingredients to produce the complete nutrition feed for breeding the livestock and poultry.

VII. Waste gas treatment:

(1) Treatment for odor fermented by the high-temperature aerobic solid fermentation system: the electromagnetic valves on the exhaust pipes of the high-temperature aerobic solid fermentation reactors are respectively opened; the odor generated by the high-temperature aerobic solid fermentation reactors during fermentation is respectively introduced into a biological deodorization filter tower by the draught fans after respectively exchanging heat by heat exchange condensers, and is discharged after being absorbed by bio-fillers in the biological deodorization filter tower and transformed to reach a standard; meanwhile, the hot air heated by the heat exchange condensers is respectively introduced into the, high-temperature aerobic solid fermentation reactors.

(2) Treatment for waste gases fermented by the cracking and propagation system: the electromagnetic valve on the exhaust pipe of each reactor is opened; the waste gases generated by the cracking and propagation system during fermentation are respectively introduced into the biological deodorization filter tower by the draught fans, and are discharged after being absorbed by the bio-fillers in the biological deodorization filter tower and transformed to reach the standard.

Beneficial effects and advantages of the present invention are analyzed as follows.

Breeding pollution of large-scale farms is treated according to a sustainable development treatment principle, an ecological cycle economic treatment principle and an automation and equipment treatment principle in the present invention.

(1) The sustainable development treatment principle is an economic growth mode which pays attention to long-term development and requires not only meeting needs of contemporary people, but also not damaging the ability of future generations to meet their needs. The system and the method provided by the present invention transform breeding wastes into resources beneficial to human beings and ecological sustainable development for constructing and repairing environments while solving a problem of pollution from the breeding wastes by treating all pollutants from the farms such as manure, urine, enclosure flushing water, sick and dead livestock and poultry and placentas, antibiotics and waste gases, so that a large-scale breeding industry cannot endanger the sustainable development of human beings.

(2) The ecological cycle economic treatment is a management mode for a whole process aiming at reducing the quantity of substances entering a production process, repeatedly using certain articles in different ways and recycling wastes, and is waste treatment meeting needs of ecological cycle and economic development; the principle is mainly reflected in three aspects of reduction, reuse and recycling. Rainwater and sewage, drinking water and sewage are separated by enclosure source separation and water-saving enclosure flushing, and the amount of sewage is reduced at source of the enclosure, thereby minimizing the feces and urine and laying a foundation for recycling the manure in the present invention. Feces and manure produced during breeding of livestock and poultry as well as the sick and dead livestock and poultry and the placentas are recycled and respectively prepared into solid and liquid organic fertilizers for being returned to the fields. Meanwhile, the feed crops such as pastures and corns are planted on the land; the feed is used to breed the livestock and poultry; the feces is used to breed the feed insects; and the generated insects are processed into the feed for feeding the livestock and poultry; thereby realizing combination of breeding and planting. The biogas generated by anaerobic fermentation of manure is reused as a fuel for treatment. The hot odor generated during treatment is recovered by heat exchange. Then, the recovered heat is used to supply heat to the reactors. The biogas generated by the anaerobic fermentation of the manure is used as a fuel for the treatment to realize resource recovery. The heat of the cracking and propagation system is recovered and reused by heat exchange. Therefore, all the treatment links form a circulating ecological closed loop, which promotes harmonious development of the breeding industry and the ecological environment.

(3) The automation and equipment treatment principle is a principle of applying an automation technology, a remote monitoring technology and automation equipment to complete waste treatment, avoid projectization and engineering of treatment work, reduce interference and influence of human factors and environmental factors, and make treatment effect and quality of finished products highly consistent. The pollutants such as the feces, the manure, the sick and dead livestock and poultry, the placentas, the odor and exhaust gases are respectively treated with the high-temperature aerobic solid fermentation reactors, the medium-temperature anaerobic liquid fermentation reactors, the cracking and propagation reactors, the biological deodorization filter tower and other devices in the present invention, to realize equipment and standardization of the treatment processes, reduce a degree of manual participation, and improve stability and continuity of the treatment processes. Meanwhile, an automatic detection and control system is utilized to realize remote real-time monitoring of equipment, processes, raw materials and products, and record destination of the pollutants in the present invention. Expert management is not required at the site due to automatic operation of the equipment and remote warning and diagnosis of the equipment are adopted. Operation conditions of the equipment at the site can be acquired from the cloud to facilitate maintenance in advance.

The feces are prepared into organic solid fertilizers by rapidly realizing harmlessness and stabilization of the materials through two stages of high-temperature aerobic fermentation and aging in the present invention. The innovation is as follows: a plurality of high-temperature thermal insulation water tanks are designed; the high-temperature thermal insulation water tanks supply heat to the materials in the high-temperature aerobic solid fermentation reactors to heat up the materials when the temperature of the materials in the high-temperature aerobic solid fermentation reactors is lower than the set temperature; the heat of the materials is absorbed by the heat exchange condensers; the heat absorbed by the heat exchange condensers is used for heating up the fresh air; then, the fresh air is introduced into the high-temperature aerobic solid fermentation reactors to provide fresh and warm air for the fermented materials, thereby not only improving heating efficiency, but also reducing the energy consumption of the system.

In view of a series of problems, such as long time, low biogas production rate, large capacity of a biogas digester, high construction cost of the biogas digester, and difficulty in cleaning biogas residues, at the bottom of the biogas digester of a traditional anaerobic fermentation method, the novel medium-temperature anaerobic liquid fermentation reactors are innovatively designed in the present invention, and the plurality of medium-temperature anaerobic liquid fermentation reactors are connected in series for medium-temperature anaerobic fermentation. The innovation is as follows. (1) The medium-temperature anaerobic fermentation is adopted to greatly improve the fermentation efficiency and shorten the fermentation time; the residence time of the materials in the biogas digester is short to lower requirements on the capacity of the biogas digester and reduce the cost of the biogas digester; meanwhile, the present solution can greatly reduce the energy consumption in comparison with a high-temperature anaerobic fermentation method. (2) The soft anaerobic fermentation bags are used as anaerobic fermentation containers to realize simple construction, greatly reduce the amount of infrastructure construction and greatly reduce infrastructure costs. (3) An inclined surface in a length direction is arranged at the bottom of each medium-temperature anaerobic liquid fermentation reactor; the lowest positions of the inclined surfaces of the plurality of medium-temperature anaerobic liquid fermentation reactors are sequentially lowered in a flow direction of the manure liquid to form the height difference; the manure liquid naturally flows through the plurality of medium-temperature anaerobic liquid fermentation reactors in sequence for multi-stage fermentation due to the height difference, to avoid the energy consumption generated by conveying the liquid to various reaction tanks in the traditional treatment method; meanwhile, the liquid in the medium-temperature anaerobic liquid fermentation reactors connected in series flows continuously; the liquid stays in a single reactor for a relatively short time; the biogas residues are slowly deposited; and the biogas residues is carried out along with the continuously flowing biogas slurry, to avoid a problem that a large amount of biogas residues are deposited and hard to be cleaned due to the materials stay in a single biogas digester too long. (4) The polarizers are arranged in the medium-temperature anaerobic liquid fermentation reactors to avoid a problem of crusting of the liquid surface of the biogas digester during traditional biogas fermentation and improve the gas production efficiency. (5) The heating coil is designed at the bottom of each reaction tank; the temperature of the materials in each medium-temperature anaerobic liquid fermentation reactor is controlled to be always stable within a limited range by injecting the circulating hot water into the heating coil of each medium-temperature anaerobic liquid fermentation reactor during fermentation, to rapidly achieve a harmlessness standard and greatly increase the fermentation efficiency; the materials stay in each multi-stage medium-temperature anaerobic liquid fermentation, reactor for more than 15 days accumulatively, are thoroughly decomposed and are stabilized, thereby reaching an agricultural standard of the biogas slurry. (6) The manure liquid is first-in-first-out in the soft anaerobic fermentation bags connected in series and cannot flow out of the soft anaerobic fermentation bags without fermentation. (7) The time that the manure liquid stays in the soft anaerobic fermentation bags can be adjusted by controlling the liquid level of the liquid outlet tank to ensure the residence time of the manure liquid for anaerobic fermentation in the soft anaerobic fermentation bags to meet a statutory time requirement.

The cracking and propagation system designed in the present invention soaks the sick and dead livestock and poultry and the placentas in the manure liquid in the closed reactors, firstly performs harmless cracking on the sick and dead livestock and poultry and the placentas at high temperature and high pressure, dissolves the sick and dead livestock and poultry and the placentas in the manure liquid to prepare the cracking liquid, then uses the cracking liquid as a culture medium of a culture solution for propagating functional microorganisms to obtain a microbial culture solution by propagation, or uses the cracking liquid as food for breeding feed insects. The innovation is as follows. (1) The pollution treatment mode is innovated in the present invention; the cracking liquid produced by cracking the sick and dead livestock and poultry and the placentas is used as the culture medium for culturing the microorganisms to culture the functional microorganisms, is respectively added into the organic solid fertilizers and the biogas slurry to prepare the bio-organic fertilizers and the microbial liquid fertilizers; the sick and dead livestock and poultry and the placentas are rich in nutrients such as protein, fat and inorganic salts; most of the nutrients are dissolved in the cracking liquid to provide necessary nutrients for growth and reproduction of the microorganisms after cracking at high temperature and high pressure; compared with a traditional chemical production method, the method provided by the present invention reduces processing costs of a post-drying step, and does not produce sewage pollution; and the propagated functional microorganisms are added into the organic fertilizers to prepare biofertilizers, thereby maximizing a resource utilization rate of the wastes and increasing an added value of the organic fertilizers. (2) The cracking and propagation reactors integrate high-temperature and high-pressure cracking and microbial propagation of the sick and dead livestock and poultry to achieve multiple purposes; considering an actual situation of high mortality rate of small livestock and poultry, the plurality of cracking reactors and propagation reactors are provided in the present invention; and a cracking process and a propagation process are separated to solve a problem that the output of sick and dead livestock and poultry is not matched with treatment capacity. (3) A plurality of aeration heads are uniformly arranged in the cracking and propagation reactors and the cracking reactors; the liquid and the sick and dead livestock and poultry are aerated from the bottom of the reactor by using high-temperature and high-pressure steam generated by the steam generators so that the cracking and propagation system is heated up and pressurized to rapidly realize harmlessness; meanwhile, a large number of bubbles generated during aeration rub the sick and dead livestock and poultry to promote rapid disintegration of the sick and dead livestock and poultry, dissolution in the manure liquid, and increase of the content of the nutrients in the manure liquid, thereby laying a material foundation for the propagation of the microbial liquid in the next step. (4) After the high-temperature and high-pressure cracking of the sick and dead livestock and poultry is completed, the hot water in the high-temperature thermal insulation water tanks and the cold water in the low-temperature thermal insulation water tanks are conveyed to the water jackets of the cracking and propagation reactors for cooling the high-temperature cracking liquid and creating a suitable temperature condition for the microbial propagation in the next step; the heated hot water is utilized to perform thermal insulation on the microbial culture solution, thereby improving the microbial propagation efficiency; and the system provided by the present invention fully recycles waste heat of the cracking liquid to avoid waste of the heat, improve heating efficiency and also reduce the energy consumption of the system. (5) Cracking residues of the sick and dead livestock and poultry are reused; the grease produced by cracking the sick and dead livestock and poultry is recovered and reused to maximize a resource conversion rate. (6) The cracking liquid is used as insect feeding food for breeding the feed insects, thereby preparing an animal-derived feed; the sick and dead livestock and poultry and the placentas are rich in nutrients such as protein, fat and inorganic salts, and are used for growth and propagation of the insects, thereby recycling the dead livestock and poultry and the placentas and also improving the insect breeding benefits and product quality.

In the present invention, the finished products such as the organic fertilizers and the bio-organic fertilizers of the pollution treatment factory are used to improve the soil of a planting base of the feed factory. The finished products such as the organic-inorganic compound liquid fertilizers and the microbial liquid fertilizers of the pollution treatment factory are used for irrigation and drip irrigation of grains, pastures and other crops planted on the supporting planting land. The grains, pastures and other crops as well as insects raised in the feed factory are used as the raw materials of the feed factory to reduce breeding costs, improve a nutrition level of the feed and promote harmonious development of the breeding industry and the ecological environment.

In conclusion, the present invention ecologically treats the pollutants such as the feces, the urine, the flushing water, the sick and dead livestock and poultry and the placentas generated during breeding of the livestock and poultry as well as the waste gases, recycles the feces, the urine, the sink and dead livestock and poultry and the placentas, not only treats all the pollutants generated during breeding of the large-scale livestock and poultry farms, but also transforms the organic wastes into usable resources, thereby having great significance to rural environmental protection, benign development of animal husbandry, and implementation of sustainable energy development strategies.

### DESCRIPTION OF THE DRAWINGS

FIG. 1 is a schematic diagram of an ecological pollution treatment system for livestock and poultry farms based on combination of planting and breeding according to the present invention;

FIG. 2 is a schematic diagram of feed insect breeding according to the present invention;

FIG. 3 is a schematic diagram of a high-temperature aerobic solid fermentation system according to the present invention;

FIG. 4 is a schematic diagram of a section of a connection relationship of a medium-temperature anaerobic liquid fermentation system according to the present invention;

FIG. 5 is a schematic diagram of a section of a structure of a medium-temperature anaerobic liquid fermentation reactor according to the present invention;

FIG. 6 is a top view of a connection relationship of a medium-temperature anaerobic liquid fermentation system according to the present invention;

FIG. 7 is a schematic diagram of planting of feed crops according to the present invention;

FIG. 8 is a schematic diagram of a cracking and propagation system according to the present invention;

FIG. 9 is a schematic diagram of connection of an aeration (steam exposure) device of a cracking and propagation reactor according to the present invention;

FIG. 10 is a schematic diagram of a cross-sectional structure of a cracking and propagation reactor according to the present invention;

FIG. 11 is a schematic diagram of a longitudinal section structure of a cracking and propagation reactor according to the present invention;

FIG. 12 is a schematic diagram of a cross-sectional structure of a sick and dead livestock and poultry conveying device according to the present invention;

FIG. 13 is a schematic diagram of a longitudinal section structure of a sick and dead livestock and poultry conveying device according to the present invention;

FIG. 14 is a schematic diagram of a structure of a propagation reactor according to the present invention;

FIG. 15 is a schematic diagram of a heating and heat balancing system according to the present invention;

FIG. 16 is a schematic diagram of a waste gas treatment system of a high-temperature aerobic solid fermentation system according to the present invention;

FIG. 17 is a schematic diagram of a waste gas treatment system of a cracking and propagation system according to the present invention;

FIG. 18 is a schematic diagram of feed insects bred with cracking liquid according to the present invention; and

FIG. 19 is a schematic diagram of an ecological cattle and sheep breeding pollution treatment system according to the present invention.

In the figures, 101—source separation pigsty; 102—pollution treatment factory, 103—feed factory, 104—high-temperature aerobic solid fermentation system, 105—medium-temperature anaerobic liquid fermentation system, 106—cracking and propagation system, 107—heating and heat balancing system, 108—waste gas treatment system, 109—detection and control system;

201—frass, 202—insects and ova, 203—animal-derived feed;

301—dry manure shed, 302—auxiliary material, 303—decomposing bacteria, 304A—high-temperature aerobic solid fermentation reactor, 304B—high-temperature aerobic solid fermentation reactor, 304M—high-temperature aerobic solid fermentation reactor, 305—aging chamber;

401—adjusting tank, 402A—medium-temperature anaerobic liquid reactor; 402B—medium-temperature anaerobic liquid reactor, 402N—medium-temperature anaerobic liquid reactor, 403—liquid outlet tank, 404—sludge pump, 405—liquid storage, tank;

501—reaction tank, 502—thermal insulation layer, 503—heat reflecting plate, 504—heating coil, 505—soft anaerobic fermentation bag, 506—drainage ditch, 507—feed port, 508—discharge port, 509—water collecting well, 510—biogas exhaust pipe, 511—thermal insulation layer, 512—pressure sensor, 513—polarizer;

601—biogas slurry, 602—fertilizer, 603—organic solid fertilizer, 604—organic-inorganic compound liquid fertilizer, 605—microbial liquid fertilizer, 606—bio-organic fertilizer, 607—supporting planting land, 608—plant-derived feed;

701—cracking and propagation reactor, 702—microbial culture solution, 703A-cracking reactor, 703B-cracking reactor, 704—propagation reactor, 705—grease;

801—support, 802—tank-body, 803—sealing cover, 804—hinge, 805—sealing ring, 806—lock, 807—water jacket, 808—aeration pipe, 809—discharge pipe, 810—circulating water inlet pipe, 811—exhaust pipe, 812—feed pipe,

813—circulating water drainage pipe, 814—electromagnetic valve, 815—aeration head; 816—guide rail, 817—temperature sensor, 818—pressure sensor, 819—safety valve, 820—thermal insulation layer;

1001—trolley, 1002—net cage, 1003—guide rail, 1004—cage body, 1005—steel wire mesh, 1006—cage cover, 1007—chain, 1008—handle, 1009—roller;

1101—atmospheric pressure hot water boiler, 1102—steam generator, 1103—solar heating system, 1104—high-temperature thermal insulation water tank, 1105—low-temperature thermal insulation water tank, 1106a—circulating water pump, 1106b—circulating water pump, 1106c—circulating water pump, 1106d—circulating water pump, 1106e—circulating water pump, 1107a—electromagnetic valve, 1107b—electromagnetic valve, 1107c—electromagnetic valve, 1107d—electromagnetic valve, 1107e—electromagnetic valve, 1107f—electromagnetic valve, 1107g—electromagnetic valve, 1107h—electromagnetic valve, 1107i—electromagnetic valve, 1107j—electromagnetic valve, 1107k—electromagnetic valve, 1107l—electromagnetic valve, 1107m—electromagnetic valve, 1107n—electromagnetic valve, 1107o—electromagnetic valve, 1107p—electromagnetic valve, 1107s—electromagnetic valve, 1107t—electromagnetic valve, 1107u—electromagnetic valve, 1107v—electromagnetic valve, 1107w—electromagnetic valve, 1107x—electromagnetic valve, 1107y-electromagnetic valve, 1110—steam conveying pipe, 1102—biogas pretreatment device;

1201a—heat exchange condenser, 1201b—heat exchange condenser, 1201m—heat exchange condenser, 1202a—draught fan, 1202b—draught fan, 1202m—draught fan, 1203—biological deodorization filter tower, 1204a—electromagnetic valve, 1204b—electromagnetic valve, 1204m—electromagnetic valve, 1205a—electromagnetic valve, 1205b—electromagnetic valve, 1205m—electromagnetic valve;

1301—aeration fan, 1302—air filter, 1303a—electromagnetic valve, 1303b—electromagnetic valve, 1303c—electromagnetic valve, 1303d—electromagnetic valve, 1304b—electromagnetic valve, 1304c—electromagnetic valve, 1304d—electromagnetic valve, 1304e—electromagnetic valve, 1305—draught fan, 1306—biological deodorization filter tower, 1307a—one-way valve, 1307b—one-way valve, and 1308—tee joint.

### DETAILED DESCRIPTION OF PREFERRED EMBODIMENTS

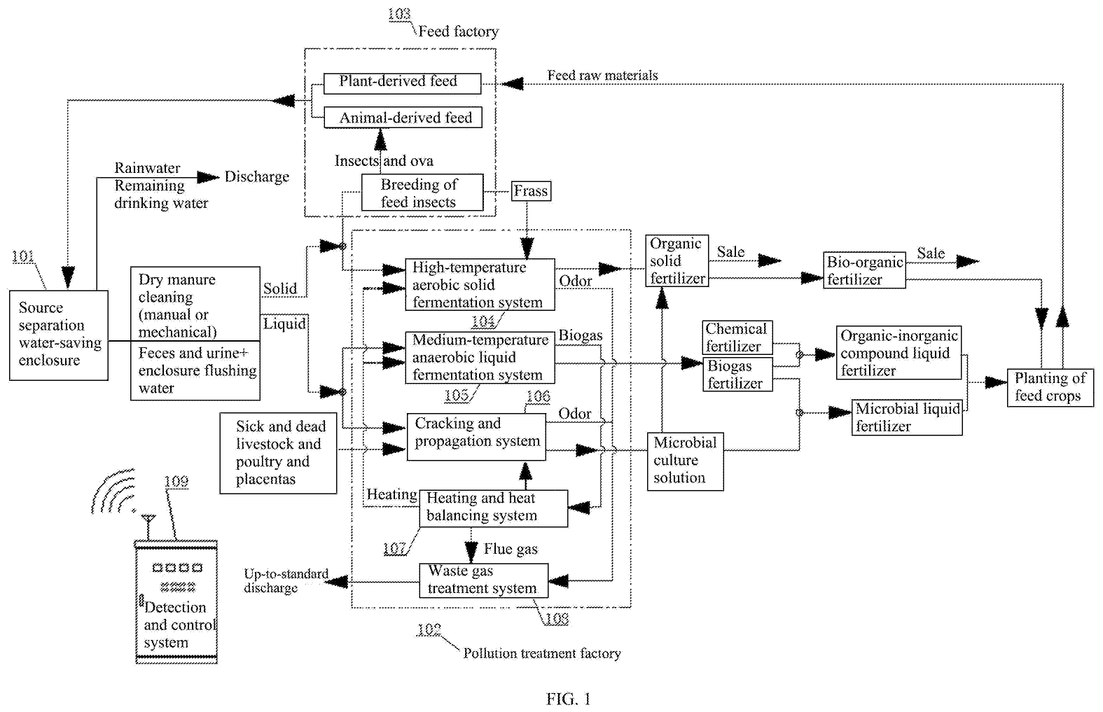

The schematic diagram of an ecological pollution treatment system for livestock and poultry farms based on combination of planting and breeding according to the present invention is shown in FIG. 1. The system includes a source separation water-saving enclosure 101, a pollution treatment factory 102, a feed factory 103 and a supporting planting land 607. The pollution treatment factory 102 and the feed factory 103 are managed by professional teams for specialized disposal of livestock and poultry manure. The pollution treatment factory 102 is specialized in transforming and preparing the livestock and poultry manure into fertilizers needed to improve soil by utilizing a supporting pollution treatment workshop at source. The feed factory 103 is specialized in transforming and preparing the livestock and poultry manure into animal-derived feed 203 and plant-derived feed 608 by utilizing the supporting planting land 607 and a feed production workshop at source. The pollution treatment factory 102 is composed of a high-temperature aerobic solid fermentation system 104, a medium-temperature anaerobic liquid fermentation system 105, a cracking and propagation system 106, a heating and heat balancing system 107, a waste gas treatment system 108 and a detection and control system 109. The source separation water-saving enclosure 101 is configured to separate rainwater and sewage, drinking water and sewage, discharge the rainwater and the remaining drinking water to outdoor ditches instead of mixing into feces and urine, prevent the rainwater and the remaining drinking water from mixing into the feces and urine at source of the enclosure by means of water-saving enclosure flushing and mechanical manure scraping or manual dry manure cleaning to minimize the feces and urine, and flush the enclosure with high-pressure spray nozzles or even high-pressure air when slaughtering or transferring the livestock and poultry. The feces cleaned by mechanical manure scraping or manual dry manure cleaning are piled up in a dry manure shed 301. Then, the feces are conveyed to the high-temperature aerobic solid fermentation system 104 and the animal-derived feed factory 103 by the manure shed 301, respectively. Frass 201 generated in the feed factory 103 is conveyed to the high-temperature aerobic solid fermentation system 104, while fecal and urinary liquid and enclosure flushing water are conveyed to an adjusting tank 404. The adjusting tank 401 is connected with a feed port of the medium-temperature anaerobic liquid fermentation system 105 and a liquid inlet of the cracking and propagation system 106. An exhaust port of fermentation odor generated by the high-temperature aerobic solid fermentation system 104 and a waste gas exhaust pipe of the cracking and propagation system 106 are connected with the waste gas treatment system 108. The heating and heat balancing system 107 is respectively connected with a heating jacket or coil of the high-temperature-aerobic solid fermentation system 104, heating coils of the medium-temperature anaerobic liquid fermentation system 105 as well as a water jacket and a coil of the cracking and propagation system 106 by pipes. Sensors of the detection and control system 109 are arranged in the above systems, to detect various key parameters. The detection and control system 109 controls connection of the above components.

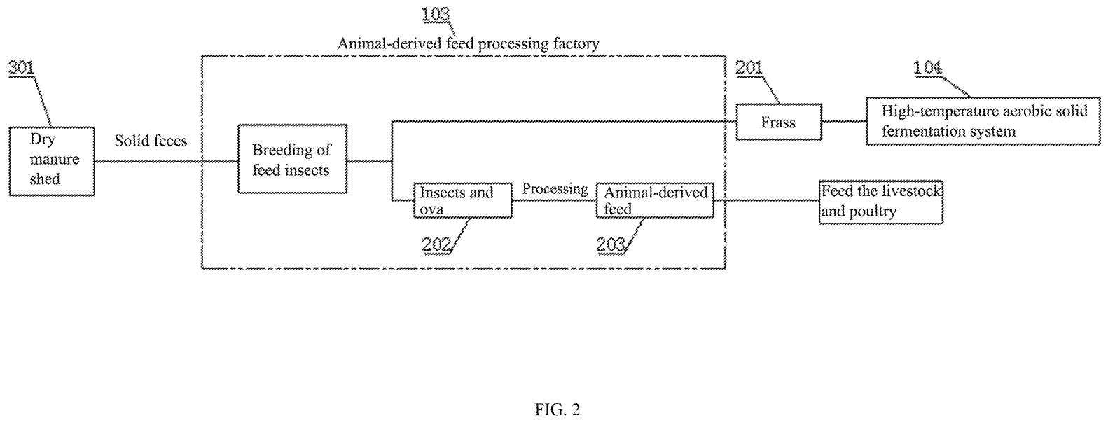

The schematic diagram of feed insect breeding according to the present invention is shown in FIG. 2. The solid feces in the dry manure shed 301 are conveyed to the feed factory 103 by the conveying device. A water content of the feces is adjusted to an appropriate range at first. Then, the feces are used as an insect feed for breeding insects to obtain insects and ova 202, which are used as an animal-derived feed 203, are mixed with a plant-derived feed 608 in a certain proportion and then are added with appropriate grains, trace elements and other ingredients to produce complete nutrition feed for breeding the livestock and poultry. Frass 201 is conveyed to high-temperature aerobic solid fermentation reactors (304A, 304B, . . . , and 304M) by the conveying device for high-temperature aerobic fermentation to obtain an organic solid fertilizer 603.

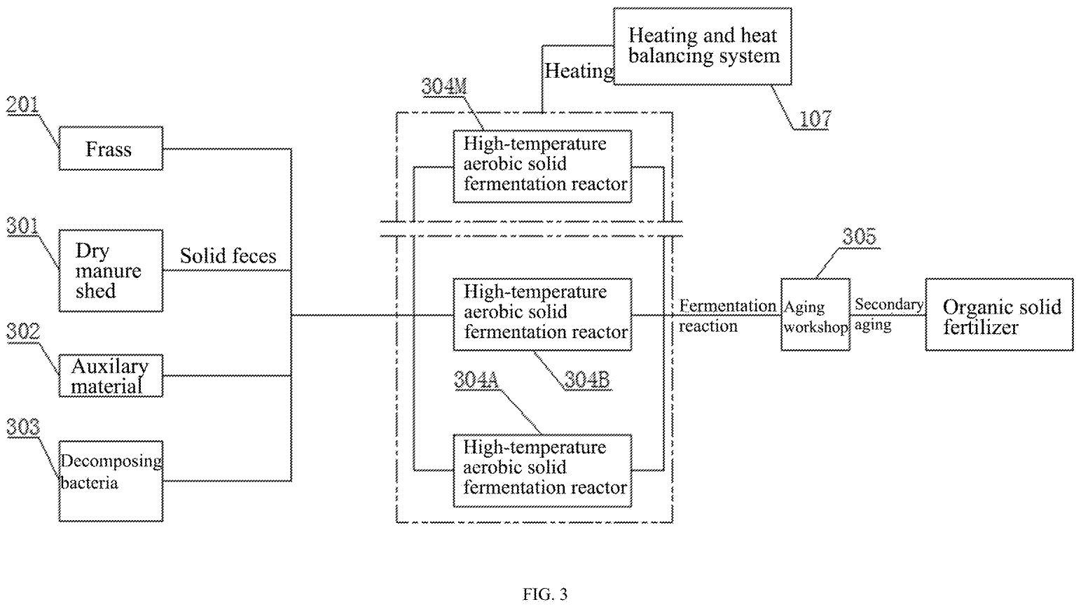

The schematic diagram of the high-temperature aerobic solid fermentation system according to the present invention is shown in FIG. 3. The high-temperature aerobic solid fermentation system 104 includes M high-temperature aerobic solid fermentation reactors (304A, 304B, . . . , and 304M). The solid feces and the frass 201 in the dry manure shed 301 as well as auxiliary materials 302 and decomposing bacteria 303 are respectively conveyed to the high-temperature aerobic solid fermentation reactors (304A, 304B, . . . , and 304M) by the conveying device. Discharge ports of the high-temperature aerobic solid fermentation reactors (304A, 304B, . . . , and 304M) are respectively connected with aging chambers 305 by the conveying devices.

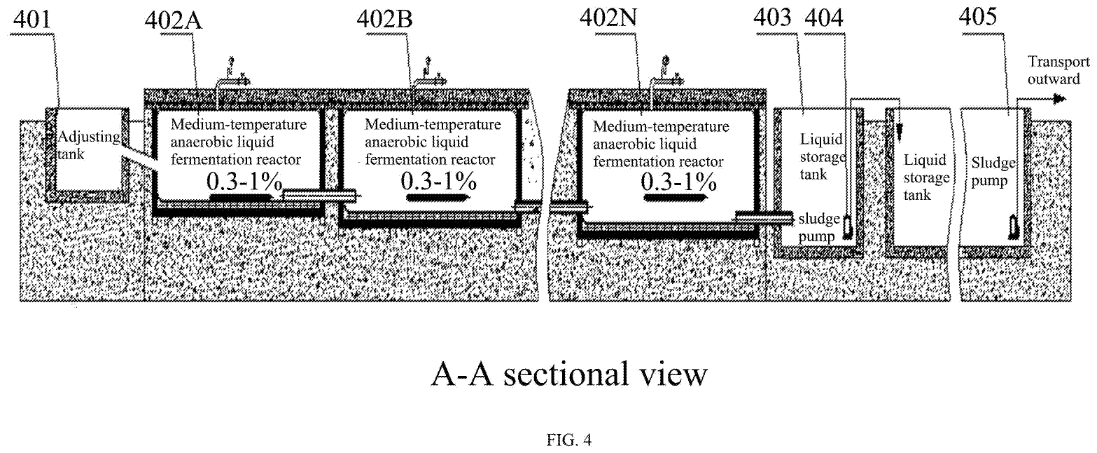

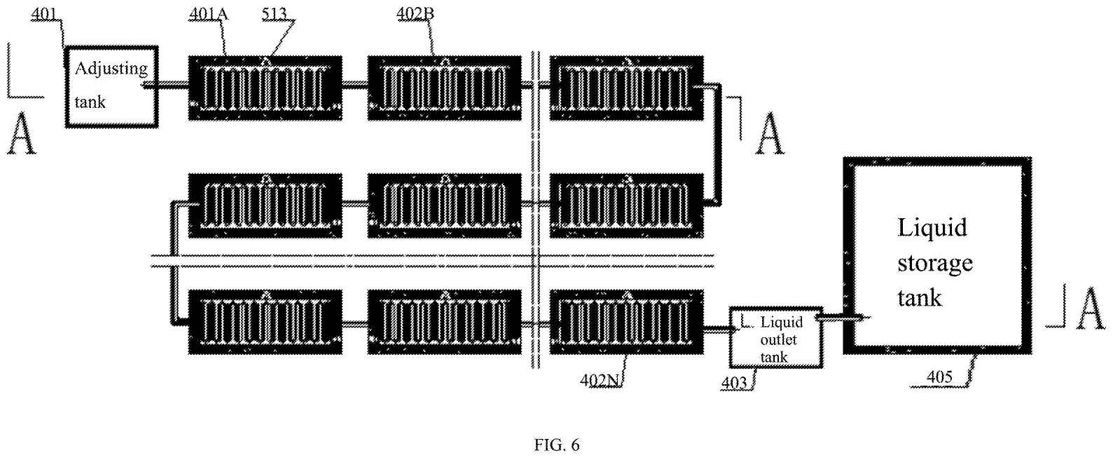

The schematic diagram of the medium-temperature anaerobic liquid fermentation system according to the present invention is shown in FIG. 4 and FIG. 6. The medium-temperature anaerobic liquid fermentation system 105 includes the adjusting tank 401, medium-temperature anaerobic liquid fermentation reactors (402A, 402B, . . . , and 402N), a liquid outlet tank 403, a sludge pump 404 and a liquid storage tank 405. A liquid outlet of the adjusting tank 401 is connected with the feed port of a first medium-temperature anaerobic liquid fermentation reactor 402A through a pipe; and the discharge port thereof is connected with the feed port of a second medium-temperature anaerobic liquid fermentation reactor 402B through the pipe, and so on, until the discharge port is connected to the feed port of the Nth medium-temperature anaerobic liquid fermentation reactor 402N; and the discharge port thereof is connected with the liquid outlet tank 403 through the pipe. Then, the liquid outlet tank 403 is connected with the liquid storage tank 405 by the sludge pump 404. For the N medium-temperature anaerobic liquid fermentation reactors (402A, 402B, . . . , and 402N), the tank bottom of the feed port of the medium-temperature anaerobic liquid fermentation reactor 402B is 0.2 m lower than the feed port of the medium-temperature anaerobic liquid fermentation reactor 402B. Similarly, the tank bottom of the Nth medium-temperature liquid fermentation reactor 402N is 0.2 m lower than the discharge port of the previous medium-temperature liquid fermentation reactor, to prevent sediments in the subsequent medium-temperature anaerobic liquid fermentation reactor from flowing back to the previous medium-temperature anaerobic liquid fermentation reactor. The tank bottom at a feed port side of each medium-temperature anaerobic liquid fermentation reactor (402A, 402B, and 402N) is higher than the tank bottom of the discharge port; and a range of inclination is 0.5% to reduce the number of sludge cleaning times of the medium-temperature anaerobic liquid fermentation reactors (402A, 402B, . . . , and 402N).

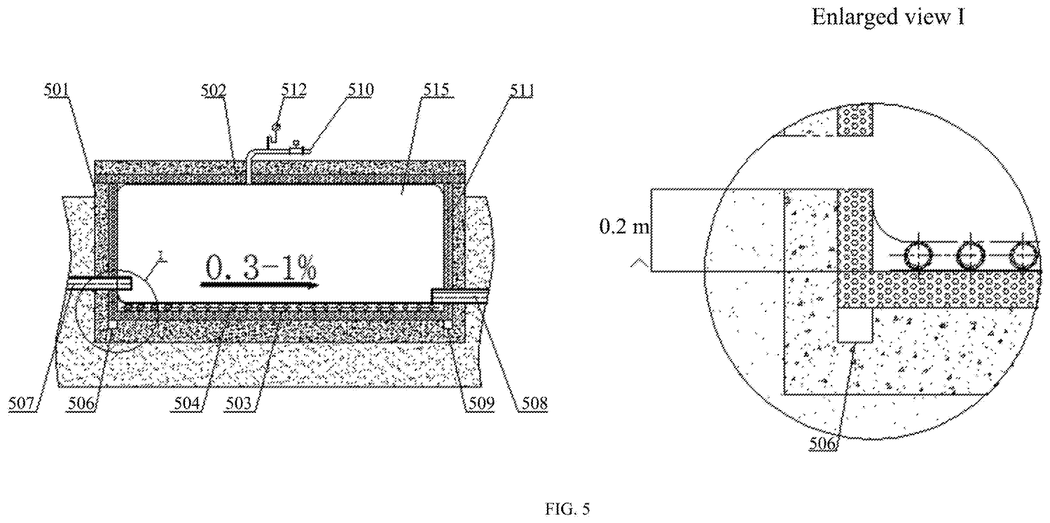

The schematic diagram of the medium-temperature anaerobic liquid fermentation reactor according to the present invention is shown in FIG. 5. The medium-temperature anaerobic liquid fermentation reactors (402A, 402B, . . . , 402N) have a structure that an inclined surface inclining from the feed port to the discharge port is constructed at the tank bottom of each rectangular reaction tank and has an inclination angle of 0.5%. A drainage ditch 506 is formed around the inclined surface. The drainage ditch 506 is connected to a water collecting well 509 at the lower ground through the pipe. Water collected at the bottoms of the reaction tanks 501 is converged to the water collecting well 509 through the drainage ditch for drainage. Thermal insulation layers 502 are arranged around the tank bottoms of the reaction tanks 501 and the inclined surface at the tank bottoms. The thermal insulation layers 502 are made of thermal insulation materials. A heat radiation plate 503 is arranged on the surface of the thermal insulation layer 502 at the tank bottom of each reaction tank 501. The heating coils 504 are uniformly fixed on the heat radiation plate 503. The heating coils 504 are covered with soft anaerobic fermentation bags 505. The feed port 507 is formed in a higher side of each soft anaerobic fermentation bag 505, and the discharge port 508 is formed in a lower side of the same in the length direction. In order to prevent liquid surfaces in the soft anaerobic fermentation bags 505 from crusting, a polarizer 513 is arranged in the middle of each rectangular reactor in the length direction; biogas exhaust pipes 510 are arranged at the tops of the soft anaerobic fermentation bags 505; pressure sensors 512 are arranged on the biogas exhaust pipes 510; and the soft anaerobic fermentation bags 505 are further covered with the thermal insulation layers 502 and waterproof covers.

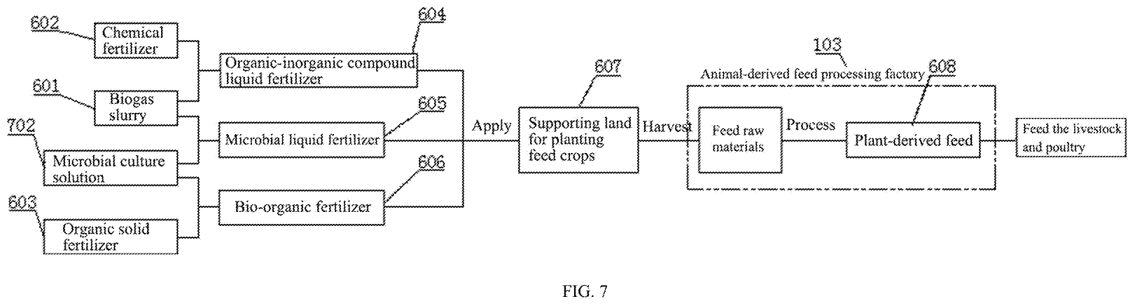

The schematic diagram of planting of feed crops according to the present invention is shown in FIG. 7. An appropriate amount of chemical fertilizer 602 and microbial culture solution 702 are respectively added into biogas slurry 601 to prepare an organic-inorganic compound liquid fertilizer 604 and a microbial liquid fertilizer 605. An appropriate amount of microbial culture solution 702 is sprayed into the organic solid fertilizer 603 and is stirred uniformly to obtain a bio-organic fertilizer 606. The organic-inorganic compound liquid fertilizer 604, the microbial liquid fertilizer 605 and the bio-organic fertilizer 606 are respectively applied to the supporting planting land 607. Feed crops are planted on the supporting planting land 607. The harvested feed raw materials are conveyed into the feed factory 103 and are processed to prepare the plant-derived feed 608 for breeding the livestock and poultry.

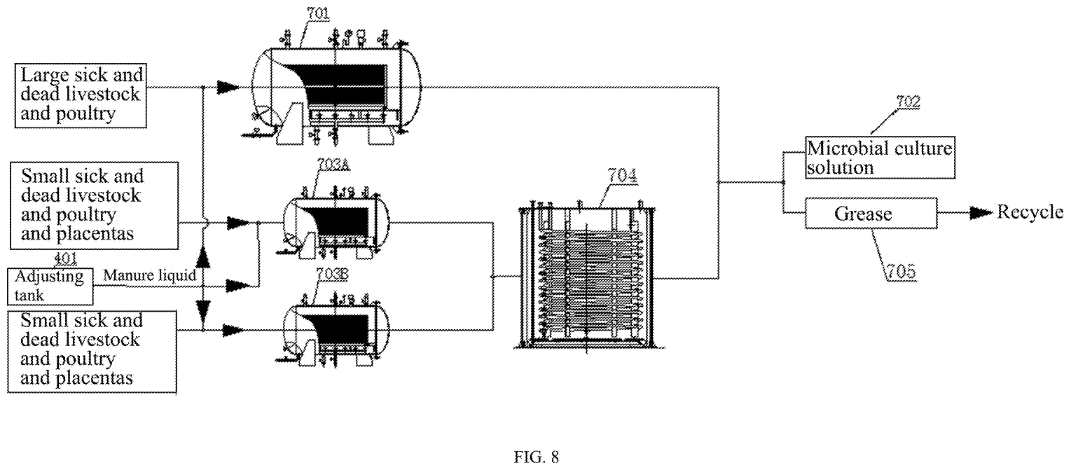

The schematic diagram of the cracking and propagation system is shown in FIG. 8. The cracking and propagation system 106 is mainly composed of cracking and propagation reactors 701, cracking reactors (703A and 703B), propagation reactors 704, steam generators 1102, aeration fans 1301, air filters 1302 and connecting pipes. The adjusting tank 401 is respectively connected to feed pipes 812 of the cracking and propagation reactors 701 and the cracking reactors (703A and 703B) by the conveying devices. Discharge pipes of the cracking reactors (703A and 703B) are connected to feed ports 905 of the propagation reactors 704 by the conveying devices. Discharge pipes 809 of the cracking and propagation reactors 701 and discharge ports 910 of the propagation reactors 704 are respectively connected to liquid storage tanks through pipes.

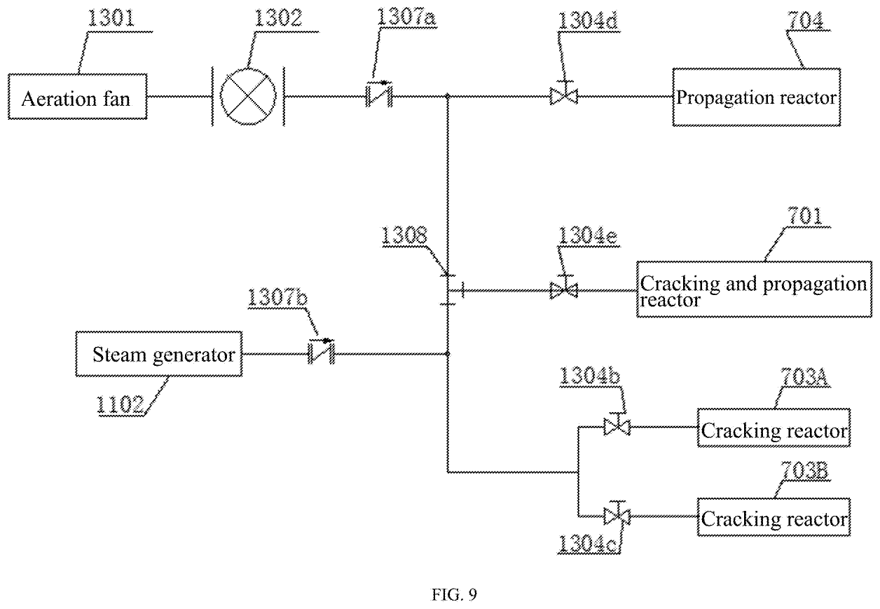

The schematic diagram of connection of an aeration (steam exposure) device of the cracking and propagation system according to the present invention is shown in FIG. 9. Steam outlets of the steam generators 1102 are respectively connected with one input port of each tee joint 1308 and steam inlets of the cracking reactors (703A and 703B) by a one-way way 1307b and a steam conveying pipe 1110. Electromagnetic valves (1304b and 1304c) are respectively arranged at front ends of steam inlet pipes of the cracking reactors (703A and 703B). Air inlet ends of the aeration fans 1301 are communicated with the atmosphere; and air outlet ends are respectively connected with another input port of each tee joint 1308 and an air inlet of the propagation reactor 704 by the air filter 1302, the one-way valve 1307a and the air conveying pipe in sequence. The electromagnetic valve 1304d is arranged on an air inlet pipe of each propagation reactor 704. An output end of the tee joint 1308 is connected with an aeration pipe 808 of the crack and propagation reactor 701 through the pipe. The electromagnetic valve 1304e is arranged on an air (steam) inlet pipe of the cracking and propagation reactor 701.

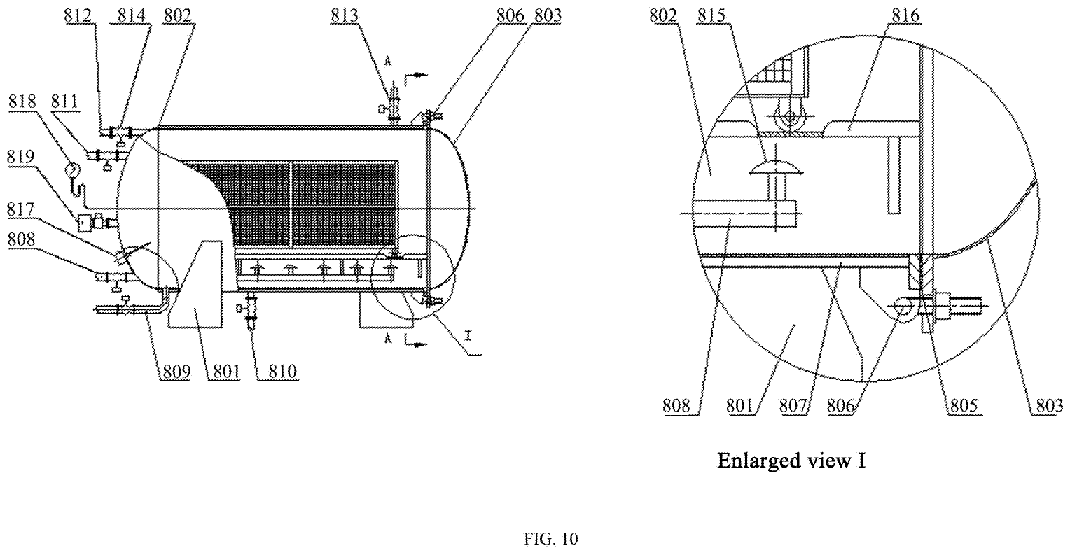

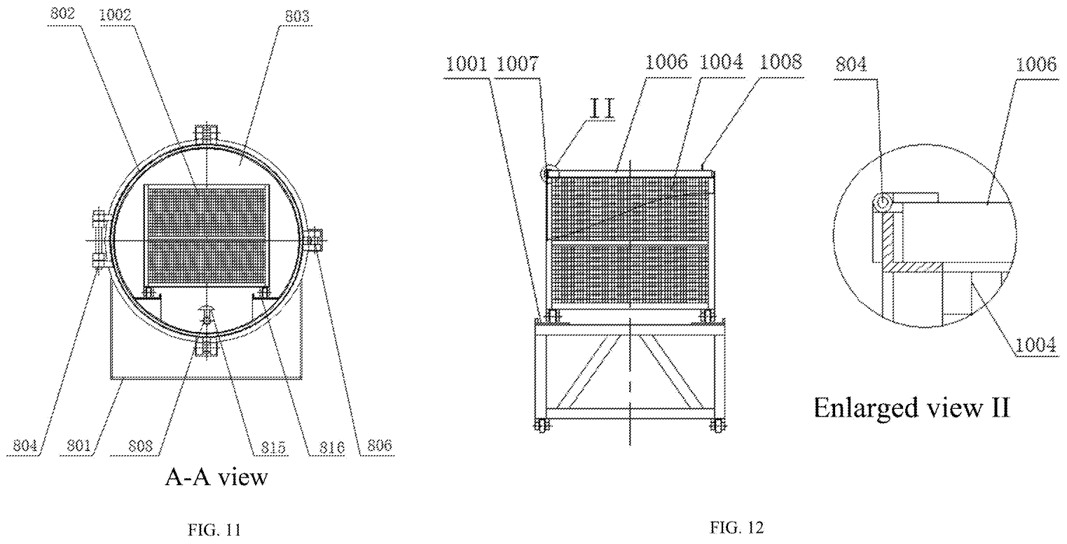

The schematic diagrams of a cross-sectional structure and a longitudinal section structure of the cracking and propagation reactor according to the present invention are shown in FIG. 10 and FIG. 11. The cracking and propagation reactor 701 is composed of a support 801, a tank body 802, a sealing cover 803 and the conveying device. The horizontally arranged tank body 802 is fixed on the support 801. One side of the tank body 802 is sealed by the sealing cover 803. A cover sealing door is mounted on the other side of the tank body 802. The cover sealing door is connected with the tank body 802 by a hinge 804. A sealing ring 805 is arranged between the cover sealing door and the tank body 802. The cover sealing door is locked and sealed with a plurality of lock 806 bolts when closed, so that a closed cracking and propagation space is formed among the sealing cover 803, the tank body 802 and the cover sealing door. Guide rails 816 parallel to an axis of the tank body 802 and radially fixed along the tank body 802 are arranged in the tank body 802 for bearing and conveying net cages 1002 carrying the sick and dead livestock and poultry and the placentas. Aeration pipes 808 and a plurality of aeration heads 815 are mounted at lower parts of the guide rails 816. An exhaust pipe 811 and a feed pipe 812 are arranged on an upper side of the sealing cover 803. A pressure sensor 818 and a safety valve 819 are further mounted on the upper side of the sealing cover 803. A temperature sensor 817 and a discharge pipe 809 are mounted on a lower side of the sealing cover. A water jacket 807 is mounted outside each horizontally arranged tank body 802, and the water jacket 807 is used for cooling each cracking and propagation reactor 701. A circulating water inlet pipe 810 of the water jacket 807 is arranged at a lower part of tank body 802. A circulating water drainage pipe 813 of the water jacket 807 is arranged at an upper part of the tank body 802. The water jacket 807 is covered with the thermal insulation layer 820 which is made of the thermal insulation material.

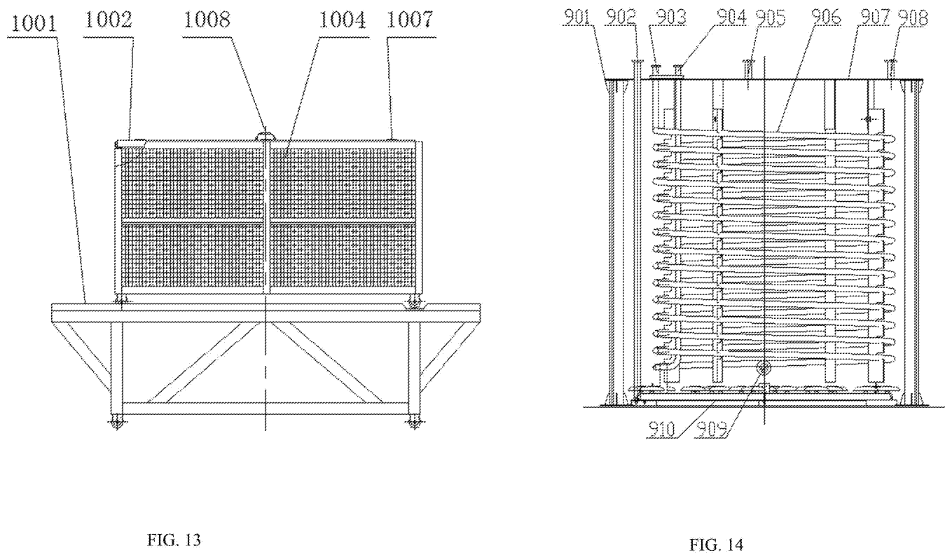

The schematic diagrams of a cross-sectional structure and a longitudinal section structure of a sick and dead livestock and poultry conveying device according to the present invention are shown in FIG. 12 and FIG. 13. The conveying device 702 is composed of a trolley 1001 and a net cage 1002. A guide rail 1003 is fixed at the upper part of the trolley 1001. The net cage 1002 is placed on the guide rail 1003. The net cage 1002 is a rectangular cage composed of a cage body 1004 and steel wire meshes 1005. The surrounding and bottom steel wire meshes 1005 are welded on the cage body 1004. A movable cage cover 1006 is arranged at the top of the cage body 1004. The cage cover 1006 is connected with the cage body 1004 by a chain 1007. A handle 1008 is further arranged outside the cage cover 1006. A plurality of rollers 1009 are fixed at the bottom of the cage body 1004. The rollers 1009 are in contact with the guide rail 1003. The direction of the guide rail 1003 on the trolley 1001 is the same as that of the guide rail 816 in the tank body 802, and is flush with the guide rail 816 in the tank body 802. When the sick and dead livestock and poultry are conveyed, the sick and dead livestock and poultry are first placed in the net cage 1002 with a forklift, and then are conveyed to a specified position in the tank body 802 by the net cage 1002 along the guide rails 816 in the trolley 1001 and the tank body 802.

The schematic diagram of a structure of the propagation reactor according to the present invention is shown in FIG. 14. The propagation reactor 704 is composed of a vertical and closed thermal insulation tank body 907, a coil 906 and an aeration device 909. An aeration port 902, a coil circulating water outlet 903, a coil circulating water inlet 904 and an exhaust port 908 are formed in the top of the tank body 907, while a discharge port 910 is formed in the bottom. The coil 906 is fixed in the tank body 907 and is immersed in the cracking liquid. The aeration device 909 is arranged at the bottom of the tank body 907. The aeration pipe extends to the outside of the tank body 907 by a tank wall and is sequentially connected with air outlets of the electromagnetic valve, the air filter and the aeration fan. The tank body 907 is covered with the thermal insulation layer made of the thermal insulation material.

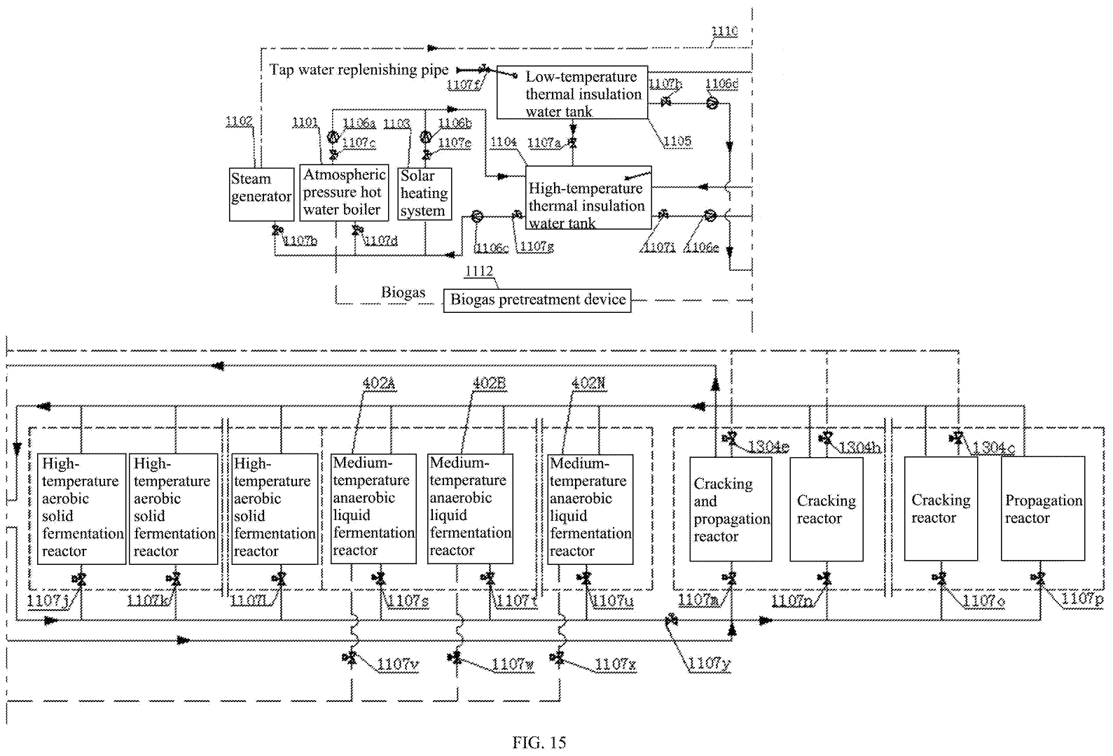

The schematic diagram of the heating and heat balancing system according to the present invention is shown in FIG. 15. The heating and heat balancing system 107 is mainly composed of an atmospheric pressure hot water boiler 1101, high-temperature thermal insulation water tanks 1104, low-temperature thermal insulation water tanks 1105, circulating water pumps (1106a-1106e), electromagnetic valves (1107a-1107y) and connecting pipes. Further, for regions with abundant solar energy resources, the heating and heat balancing system 107 also includes a solar heating system 1103. The high-temperature water tanks 1104 are water sources of the atmospheric pressure hot water boiler 1101, the solar heating system 1103 and the steam generator 1102. The high-temperature water tanks 1104 respectively convey hot water to the atmospheric pressure hot water boiler 1101, the solar heating system 1103 and the steam generator 1102 through water outlet pipes 1108. The water heated by the atmospheric pressure hot water boiler 1101 and the solar heating system 1103 is sent back to the high-temperature water tanks 1104 through respective pipes for realizing energy storage. The other water outlet pipe 1111 of each high-temperature water tank 1104 is connected with the circulating water pump 1106e to respectively convey the hot water to the high-temperature aerobic solid fermentation reactors (304A, 304B, . . . , and 304M), the medium-temperature anaerobic liquid fermentation reactors. (402A, 402B, . . . , and 402N), the cracking and propagation reactors 701, the cracking reactors (703A and 703B) and the propagation reactors 704. Return water of each reactor is sent back to each high-temperature water tank 1104 through respective water returning pipes. The low-temperature water tanks 1105 are water sources of the high-temperature water tanks 1104. The low-temperature water tanks 1105 are arranged above the high-temperature water tanks 1104 and automatically replenish water to the high-temperature water tanks 1104 under control of the detection and control system 109. The other water outlet pipe of each low-temperature water tank 1105 is respectively connected with the water jackets 807 of the cracking and propagation reactors 701 and the cracking reactors (703A and 703B) and the coils 906 of the propagation reactors 704 by the circulating water pumps 1106e. The water is sent back to the low-temperature water tanks 1105 through respective water returning pipes to realize circulation. A biogas exhaust pipe 510 at the top of each soft biogas bag of the medium-temperature anaerobic liquid fermentation reactors (402A, 402B, . . . , and 402N) is connected with a biogas pretreatment device 1112 in parallel through an exhaust pipe. The biogas in the biogas pretreatment device 1112 is treated and purified by the pretreatment device and then is conveyed to the atmospheric pressure hot water boiler 1101 for combustion to supply heat. Electromagnetic valves (1107v, 1107w and 1107x) are arranged on each exhaust pipe.

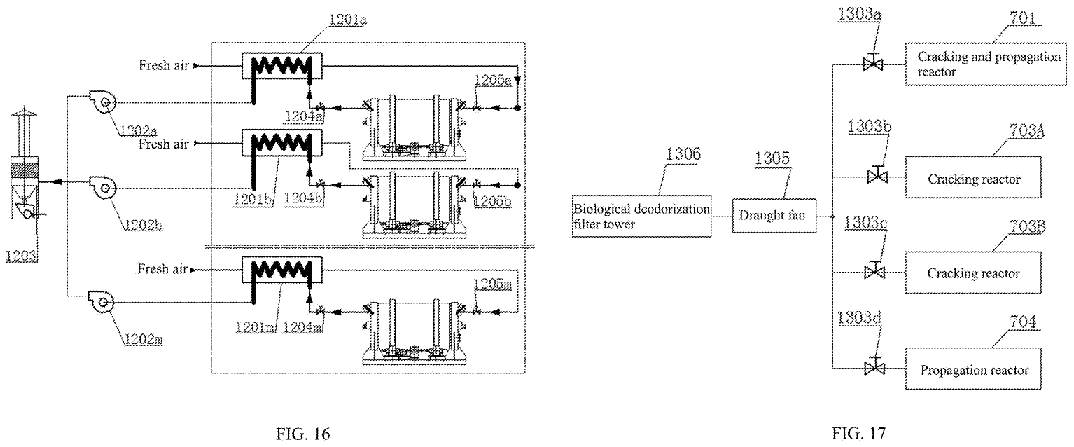

The schematic diagram of the waste gas treatment system 108 of the high-temperature aerobic solid fermentation system 104 according to the present invention is shown in FIG. 16. The waste gas treatment system has a structural connection as follows. The exhaust ports of the high-temperature aerobic solid fermentation reactors (304A, 304B, . . . , and 304M) are respectively connected with waste gas inlets of heat exchange condensers (1201a, 1201b, . . . , and 1201m) through pipes. A waste gas outlet of each heat exchange condenser (1201a, 1201b, . . . , and 1201m) is respectively connected with an-input end of a draught fan (1202a, 1202b, . . . , and 1202m) through the pipe. Air inlets of the heat exchange condensers (1201a, 1201b, . . . , and 1201m) are communicated with atmosphere. Air outlets of the heat exchange condensers (1201a, 1201b, . . . , and 1201m) are respectively connected with the gas inlets of the high-temperature aerobic solid fermentation reactors (304A, 304B, . . . , and 304M) through the pipes. Electromagnetic valves (1204a, 1204b, . . . , and 1204m) and (1205a, 1205b, . . . , and 1205m) are respectively arranged on intake pipes and exhaust pipes of the high-temperature aerobic solid fermentation reactors (304A, 304B, . . . , and 304M). An output end of each draught fan (1202a, 1202b, . . . , and 1202m) is respectively connected to a gas inlet of a biological deodorization filter tower 1203 in parallel through the pipe. An exhaust port of the biological deodorization filter tower 1203 is communicated with the atmosphere through a vertical pipe.

The schematic diagram of the waste gas treatment system 108 of the cracking and propagation system 106 according to the present invention is shown in FIG. 17. The waste gas treatment system has a structural connection as follows. Exhaust pipes of the cracking and propagation reactors 701, the cracking reactors (703A and 703B) and the propagation reactors 704 are respectively connected to input ends of the draught fans 1305. Electromagnetic valves (1303a, 1303b, 1303c and 1303d) are respectively arranged on the exhaust pipe of each reactor. The output ends of the draught fans 1305 are connected with gas inlets of the biological deodorization filter towers 1306 in parallel through the pipes. The exhaust ports of the biological deodorization filter towers 1306 are communicated with the atmosphere through the vertical pipes.

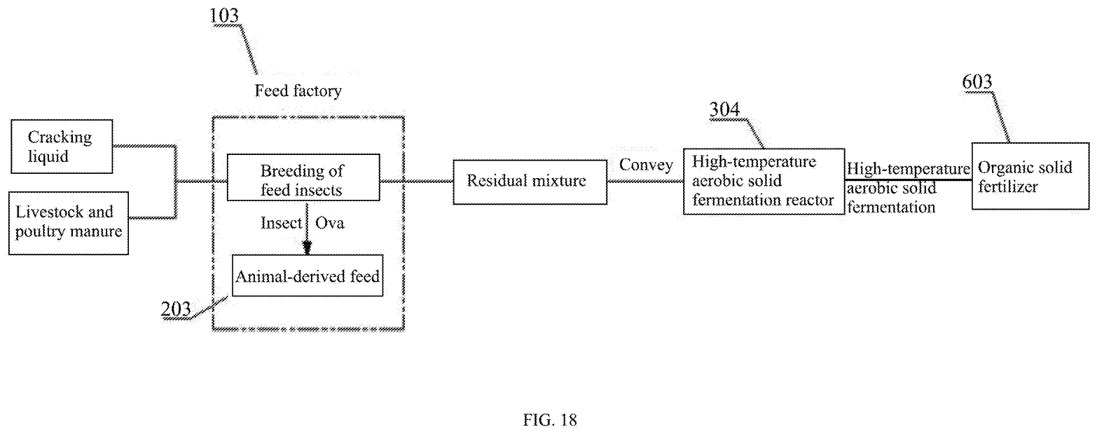

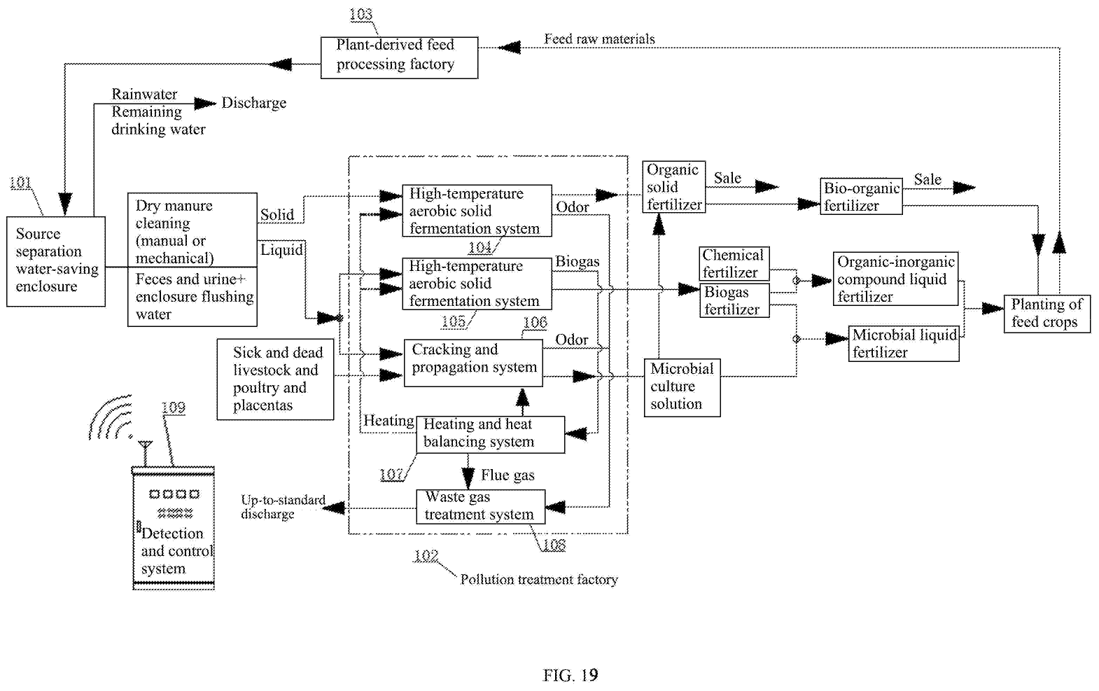

The schematic diagram of feed insects bred with cracking liquid according to the present invention is shown in FIG. 18. The livestock and poultry feces is added into the cracking liquid and mixed uniformly. A mixture is used as an insect feed for breeding insects to obtain insects and ova 202, which are used as an animal-derived feed 203, are mixed with a plant-derived feed 608 in a certain proportion and then are added with appropriate grains, trace elements and other ingredients to produce complete nutrition feed for breeding the livestock and poultry. <2> The residual mixture of the cracking liquid and the livestock and poultry feces as well as frass are conveyed to the high-temperature aerobic solid fermentation reactors (304A, 304B, . . . , and 304M) by the conveying device for high-temperature aerobic fermentation to obtain an organic solid fertilizer 603.

### EMBODIMENT 1

An ecological pollution treatment method for pig farms based on combination of planting and breeding includes:

I. Source separation and water saving of enclosure: rainwater and sewage, drinking water and sewage are separated by the source separation water-saving enclosure 101; the rainwater and the remaining drinking water are discharged to ditches outside the enclosure instead of being mixed into feces and urine; water-saving enclosure flushing and mechanical manure scraping or manual dry manure cleaning are adopted to prevent the rainwater and the remaining drinking water from mixing into the feces and urine of pigs at source of the enclosure and minimize the feces and urine; the enclosure is cleaned with high-pressure spray nozzles or even high-pressure air; the pig feces cleaned by mechanical manure scraping or manual dry manure cleaning is piled up in a dry manure shed 301; then, the feces is conveyed to the high-temperature aerobic solid fermentation system 104 and the animal-derived feed factory 103 by the manure shed 301, respectively; and the feces and urine of the pigs and enclosure flushing water are conveyed to the adjusting tank 401.

II. Breeding of black soldier fly: a water content of the pig feces is adjusted to an appropriate range at first; then, the feces is inoculated with an appropriate amount of black soldier fly larvae of right age, to obtain a mixture of frass 201 and black soldier flies and ova 202 after growth and propagation for a period of time; the black soldier flies and ova 202 are dried and crushed to obtain a black soldier fly feed 203, which is mixed with the plant-derived feed 608 in a certain proportion and then is added with appropriate grains, trace elements and other ingredients to produce complete nutrition feed for breeding the pigs; and frass 201 is conveyed to the high-temperature aerobic solid fermentation reactors (304A, 304B, . . . , and 304M) by the conveying device for high-temperature aerobic fermentation to obtain the organic solid fertilizer 603.

III. High-temperature aerobic fermentation of feces:

(1) The electromagnetic valve 1107f is opened so that tap water is automatically replenished to the low-temperature thermal insulation water tank 1105; the electromagnetic valve 1107a is opened so that the low-temperature thermal insulation water tank 1105 automatically replenishes the water to the high-temperature thermal insulation water tank 1104 by utilizing a height difference; the electromagnetic valves (1107g, 1107b and 1107d) are opened; the circulating water pumps (1106a and 1106b) are started; the atmospheric pressure hot water boiler 1101 and the solar heating system 1103 are started; the water of the high-temperature thermal insulation water tank 1104 is conveyed to the atmospheric pressure hot water boiler 1101 and the solar heating system 1103 for heating; then, the electromagnetic valves (1107c and 1107e) are opened; the circulating water pumps (1106a and 1106b) are started; the hot water is conveyed to the high-temperature thermal insulation water tank 1104 for energy storage; the electromagnetic valve 1107i is opened; the electromagnetic valve (1107j, 1107k or 1107l) at the front end of the heating jacket or coil of the high-temperature aerobic solid fermentation reactor (304A, 304B or 304M) is opened; the hot water circulating pump 1106e is started; and the hot water is conveyed to heat up materials in the high-temperature aerobic solid fermentation reactor (304A, 304B or 304M).

(2) The solid feces and the auxiliary materials 302 in the dry manure shed 301 as well as the decomposing bacteria 303 are conveyed into the high-temperature aerobic solid fermentation reactor (304A, 304B or 304M) by the conveying device; the water content of the mixture is controlled at 55-65%; and the detection and control system 109 simultaneously starts a driving device of the high-temperature aerobic solid fermentation reactor (304A, 304B or 304M) while adding the materials, thereby realizing feeding and stirring.

(3) After completing feeding, the detection and control system 109 controls the high-temperature aerobic solid fermentation reactor (304A, 304B or 304M) to stop stiffing for 50 min, stir for 10 min, stop stirring for 50 min, and then stir for 10 min; a cycle of stop-stir-stop-stir-stop-stir is a timed stirring program; and meanwhile, the detection and control system 109 automatically starts the draught fan (1202a, 1202b or 1202m) to supply oxygen to fermented materials in the high-temperature aerobic solid fermentation reactor (304A, 304B or 304M) when the high-temperature aerobic solid fermentation reactor (304A, 304B or 304M) stirs. <1> The hot water enters the heating jacket or coil to rise the temperature of the materials in the high-temperature aerobic solid fermentation reactor (304A, 304B or 304M) when the detection and control system 109 detects that the temperature of the materials in the high-temperature aerobic solid fermentation reactor (304A, 304B or 304M) is lower than a set temperature 60° C. of the materials. <2> The timed stirring program is stopped and changed to a temperature-controlled stirring program when the temperature of the materials in the high-temperature, aerobic solid fermentation reactor (304A, 304B or 304M) is greater than or equal to 70° C.; the draught fan (1202a, 1202b or 1202m) is started, the high-temperature aerobic solid fermentation reactor (304A, 304B or 304M) is driven to stir; the timed stirring program is not started until the temperature of the materials in the high-temperature aerobic solid fermentation reactor (304A, 304B or 304M) is lower than 70° C.; the temperature of the materials in the high-temperature aerobic solid fermentation reactor (304A, 304B or 304M) is maintained in 60-70° C.; and the timed stirring program and the temperature-controlled stirring program of the high-temperature aerobic solid fermentation reactor (304A, 304B or 304M) are adopted to establish an appropriate fermentation temperature and provide sufficient oxygen for the materials in the high-temperature aerobic solid fermentation reactor (304A, 304B or 304M) and establish an appropriate environment for high-temperature aerobic fermentation of feces solids.

(4) The high-temperature aerobic fermentation is completed in 24 h after feeding the materials; the detection and control system 109 controls the high-temperature solid fermentation reactor (304A, 304B or 304M) to stop, immediately feed 50% of materials after discharging 50% of the materials, and then immediately feed 50% of materials after discharging 50% of the materials every 24 h; similarly, a spiral discharge machine is started at first when discharging the materials; and meanwhile, the detection and control system 109 controls the high-temperature solid fermentation reactor (304A, 304B or 304M) to stir and guide the discharge.

(5) The materials discharged from the high-temperature aerobic solid fermentation reactor (304A, 304B or 304M) are conveyed to the aging chamber 305 by the conveying device; the materials are regularly turned or aerated in this period so that the materials are cooled and lose water until the materials are completely decomposed to prepare an organic fertilizer.

(6) The detection and control 109 respectively detects the temperature of the materials in each high-temperature aerobic solid fermentation reactor (304A, 304B or 304M) when the high-temperature aerobic solid fermentation reactors (304A, 304B and 304M) simultaneously ferment, so that the temperature of the materials in each high-temperature aerobic solid fermentation reactor (304A, 304B or 304M) is maintained in 60-70° C.

(7) The detection and control system 109 detects and controls the water temperature in the high-temperature thermal insulation water tank 1104 to keep the water temperature constant in 70-85° C. <1> The atmospheric pressure hot water boiler 1101 is started when the temperature in the high-temperature. thermal insulation water tank 1104 is lower than 70° C.; and the circulating pump 1106e at the output end of the solar heating system 1103 is started to convey the hot water to the high-temperature thermal insulation water tank 1104 when the temperature of the hot water in the heat collecting water tank of the solar heating system 1103 is greater than 70° C.; and <2> the atmospheric pressure hot water boiler is shut down when the temperature in the high-temperature thermal insulation water tank 1104 reaches 85° C.

IV. Medium-temperature anaerobic fermentation of manure liquid:

(1) The feces and urine and the enclosure flushing water are conveyed into the adjusting tank 401, so that a liquid level of the manure liquid in the adjusting tank 401 keeps rising; the manure liquid naturally flows into the first medium-temperature anaerobic liquid fermentation reactor 402A along a connecting pipe due to a height difference when the liquid level is higher than a liquid outlet of the adjusting tank 401; the detection and control system 109 controls to open the electromagnetic valve 1107s at the front end of the heating coil of the first medium-temperature anaerobic liquid fermentation reactor 402A; the hot water enters the heating coil 504 of the first medium-temperature anaerobic liquid fermentation reactor 402A for circulation to rapidly rise the temperature of the materials in the first medium-temperature anaerobic liquid fermentation reactor 402A to 35° C.; and the materials start to perform a medium-temperature anaerobic fermentation reaction.

(2) The manure liquid naturally flows into the second medium-temperature anaerobic liquid fermentation reactor 402B along the connecting pipe due to the height difference when the liquid level of the liquid in the first medium-temperature anaerobic liquid fermentation reactor 402A is higher than the liquid outlet; the detection and control system 109 controls to open the electromagnetic valve 1107t at the front end of the heating coil of the second medium-temperature anaerobic liquid fermentation reactor 402B, so that the hot water enters the heating coil of the second medium-temperature anaerobic liquid fermentation reactor 402B for circulation to rapidly rise the temperature of the materials to 35° C., and the materials keep performing the medium-temperature anaerobic fermentation reaction.

(3) The manure liquid naturally flows out along the connecting pipe due to the height difference when the liquid level of the liquid in the second medium-temperature anaerobic liquid fermentation reactor 402B is higher than the liquid outlet, and so on, until the manure liquid naturally flows into the Nth medium-temperature anaerobic liquid fermentation reactor 402N; the detection and control system 109 controls to open the electromagnetic valve 1107u at the front end of the heating coil 504 of the Nth medium-temperature anaerobic liquid fermentation reactor 402N, so that the hot water enters the heating coil 504 of the Nth medium-temperature anaerobic liquid fermentation reactor 402N for circulation to rapidly rise the temperature of the materials to 35° C., and the materials keep performing the medium-temperature anaerobic fermentation reaction.

(4) Polarizers 513 in the medium-temperature anaerobic liquid fermentation reactors (402A, 402B, and 402N) are started in a time-division manner, to prevent the liquid surface of the liquid from “crusting” and slow down sedimentation, of liquid sediments,

(5) The detection and control system 109 respectively detects and controls the temperature of the materials in the medium-temperature anaerobic liquid fermentation reactors (402A, 402B, . . . , and 402N) to keep the water temperature constant within a range of 35-50° C.; the manure liquid sequentially flows through N medium-temperature anaerobic liquid fermentation reactors (402A, 402B, . . . , and 402N); and fermentation liquid in the Nth medium-temperature anaerobic liquid fermentation reactor 402N naturally flows into the liquid outlet tank 403 along the pipe due to the height difference, to prepare a biogas fertilizer.

(5) The detection and control system 109 regularly starts the sludge pump 404 to control the liquid level of the liquid outlet tank 403 according to a set anaerobic fermentation time, and ensures the manure liquid to stay in the medium-temperature anaerobic liquid fermentation reactors (402A, 402B, and 402N) for more than 15 days; the biogas slurry in the liquid outlet tank 403 is pumped into the liquid storage tank 405 by the sludge pump 404 so that the liquid level of the liquid in the liquid outlet tank 403 is lowered after the time for medium-temperature anaerobic fermentation reaches 15 days; and the sludge pump 404 is turned off after the detection and control system 109 detects that the liquid level of the liquid outlet tank reaches a lower limit of the liquid level.

(6) The biogas generated by the medium-temperature anaerobic liquid fermentation reactors (402A, 402B, . . . , and 402N) is conveyed to the biogas pretreatment device 1112 through the biogas exhaust pipe 510 and the conveying pipe for treatment, and then is used as a combustion fuel of the steam generator 1102. The steam generator 1102 also uses electricity, diesel and biomass as supplementary fuels when the temperature is low in winter.

V. Cracking and propagation of sick and dead pigs and placentas:

(1) Cracking of sick and dead pigs and placentas:

<1> A forklift or other transfer equipment is used to place large sick and dead pigs into the net cage 1002; the net cage 1002 is pushed into the cracking and propagation reactors 701 by the conveying device; the forklift or other transfer equipment is used to place the small sick and dead pigs and placentas into the net cage 1002; the net cage 1002 is pushed into the cracking reactor (703A or 703B) by the conveying device; the cover sealing door is closed; and the liquid in the adjusting tank 401 is conveyed into the cracking and propagation reactors 701 and the cracking reactor (703A or 703B) by the conveying pumps so that the net cages 1002 are semi-immersed in the manure liquid.

<2> The electromagnetic valve 1107b on the water inlet pipe of the steam generator is opened; the hot water in the high-temperature thermal insulation water tank 1104 is pumped into the steam generator 1102; then, the steam generator 1102 is started; finally, the electromagnetic valve (1304e, 1304b or 1304c) at the front ends of the steam inlet pipes of the cracking and propagation reactors 701 and the cracking reactor (703A or 703B) is respectively opened; hot steam generated by the steam generator 1102 is respectively conveyed into the cracking and propagation reactors 701 and the cracking reactor (703A or 703B) so that the temperature and the pressure of the liquid in the cracking and propagation reactors 701 and the cracking reactor (703A or 703B) are increased; the exhaust valves of the cracking and propagation reactors 701 and the cracking reactor (703A or 703B) are respectively closed after exhausting cold air in the cracking and propagation reactors 701 and the cracking reactor (703A or 703B), so that the temperature and the pressure in the cracking and propagation reactors 701 and the cracking reactor (703A or 703B) respectively reach 130° C. and 0.25 Mpa; the sick and dead pigs and the placentas start to be cracked at high temperature and high pressure; and the detection and control system 109 detects and controls the temperature and the pressure in the cracking and propagation reactors 701 and the cracking reactor (703A or 703B) to keep them constant within 130-140° C. and 0.25-0.35 Mpa for more than 30 min, so that the sick and dead pigs are completely harmless, and are disintegrated and dissolved in the liquid to obtain the cracking liquid.

<3> The steam generator 1102 is turned off after high-temperature and high-pressure cracking is completed; the electromagnetic valve 1107y is opened; the electromagnetic valve (1107m, 1107n or 1107o) on steam inlet pipes of the cracking and propagation reactors 701 and the cracking reactor (703A or 703B) is opened; the electromagnetic valve 1107i is opened; the circulating water pump 1106e is started; the hot water enters the water jackets of the cracking and propagation reactors 701 and the cracking reactor (703A or 703B) for circulation to balance the temperature of the cracking liquid with the temperature of the hot water of the high-temperature thermal insulation water tank 1104; then, the circulating water pump 1106e and the electromagnetic valve 1107i on the water outlet pipe of the high-temperature thermal insulation water tank 1104 are closed; the electromagnetic valve 1107y is closed; the circulating water pump 1106d and the electromagnetic valve 1107h on the water outlet pipe of the low-temperature thermal insulation water tank 1105 are started; cold water enters the water jackets of the cracking and propagation reactors 701 and the cracking reactor (703A or 703B) for circulation so that the cracking liquid is cooled to 25-35° C.; and the circulating water pump 1106d and the electromagnetic valve 1107h are closed.

(2) Propagation of the cracking liquid

<1> The electromagnetic valve on the discharge pipe of the cracking reactor (703A or 703B) is opened; the cracking liquid in the cracking reactor (703A or 703B) is conveyed into the propagation reactor 704; pre-cultured microbial seed liquid is respectively conveyed into the cracking, and propagation reactor 701 and the propagation reactor 704 through the feed pipe 812 of the cracking and propagation reactor 701 and the feed port 905 of the propagation reactor 704; the aeration fan 1301. is started; the detection and control system 109 respectively controls opening or closing of the electromagnetic valve 1304d and the electromagnetic valve 1304e; sterile air filtered by the air filter 1302 is respectively conveyed into the cracking and propagation reactor 701 and the propagation reactor 704; meanwhile, the detection and control system 109 detects and controls the temperature in the cracking and propagation reactor 701 and the propagation reactor 704 to respectively keep the temperature in 25-35° C.; and a detection and control method includes: 1) the detection and control system 109 controls to turn on the circulating water pump 1106e on the water outlet pipe of the high-temperature thermal insulation water tank 1104, the electromagnetic valve 1107i is opened, and the electromagnetic valve (1107m or 1107p) is opened when the detection and control system 109 detects that the temperature in the cracking and propagation reactor 701 or the propagation reactor 704 is lower than 25° C.; the circulating water pump 1106e is stopped, the electromagnetic valve 1107i is closed; and the electromagnetic valve (1107m or 1107p) is closed when the culture solution in the cracking and propagation reactor 701 and the propagation reactor 704 is heated up to 35° C.; 2) the detection and control system 109 controls to start the aeration fan 1301, and the electromagnetic valve (1304d or 1304e) is opened to aerate the cracking and propagation reactor 701 or the propagation reactor 704 when the detection and control system 109 detects that the temperature in the cracking and propagation reactor 701 or the propagation reactor 704 exceeds 35° C.; the aeration fan 1301 is turned off, and the electromagnetic valve (1304d or 1304e) is closed when the materials in the cracking and propagation reactor 701 or the propagation reactor 704 is cooled to 25-35° C.

<2> A propagation process is completed when the concentration of a culture bacteria solution reaches requirements after the cracking liquid is cultured for 3 days; the discharge pipe 809 of the cracking and propagation reactor 701 is respectively opened to exchange heat with the discharge pipe 910 at the bottom of the propagation reactor 704; the culture solution is discharged and stored in the liquid storage tank, and then is separated by an oil-water separator to obtain the microbial culture solution 702 and grease 705; and the grease 705 is used as an industrial raw material.

<3> The detection and control system 109 respectively detects and controls the temperature and the pressure of the materials in each reactor according to different cracking and propagation stages when a plurality of cracking and propagation reactors 701, the cracking reactors (703A and 703B) and the propagation reactor 704 react at the same time, so that the temperature and the pressure in each reactor are maintained within the set ranges.

VI. Planting of feed crops:

(1) The microbial culture solution 702 is sprayed to the aged organic solid fertilizer 603 in a certain proportion, and are stirred uniformly to obtain a bio-organic fertilizer 606; the microbial culture solution 702 is added into the biogas slurry 601 in a certain proportion to obtain a microbial liquid fertilizer 605; and an appropriate amount of NPK fertilizer 602 is added into the biogas slurry to prepare an organic-inorganic compound liquid fertilizer 604 according to growth demands of Pennisetum hydridum.

(2) An appropriate amount of organic solid fertilizer 603, bio-organic fertilizer 606, microbial liquid fertilizer 605 and organic-inorganic compound liquid fertilizer 604 are respectively applied according to the growth demands of the feed crops before and during planting of the Pennisetum hydridum according to the breeding stock of a pig farm and the supporting planting land 607 with the assimilative capacity of the Pennisetum hydridum; the Pennisetum hydridum is harvested and conveyed to the plant-derived feed factory 103 and is processed to prepare the plant-derived feed 608, which is mixed with the black soldier fly feed 203 in a certain proportion and then is added with appropriate grains, trace elements and other ingredients to produce the complete nutrition feed for breeding the pigs.

VII. Waste gas treatment:

(1) Treatment for odor fermented by the high-temperature aerobic solid fermentation system 104: the electromagnetic valve (1205a, 1205b, or 1205m) on the exhaust pipe of the high-temperature aerobic solid fermentation reactor (304A, 304B, . . . , or 304M) is respectively opened; the odor generated during fermentation is respectively introduced into the biological deodorization filter tower 1203 by the draught fan (1202a, 1202b, . . . , or 1202m) after respectively exchanging heat by the heat exchange condenser (1201a, 1201b, . . . , or 1201m) and is discharged after being absorbed by bio-fillers in the biological deodorization filter tower 1203 and transformed to reach a standard; meanwhile, the fresh air heated by the heat exchange condenser (1201a, 1201b, . . . , or 1201m) is respectively introduced into the high-temperature aerobic solid fermentation reactor (304A, 304B, . . . , or 304M).

(2) Treatment for waste gases fermented by the cracking and propagation system 106: the electromagnetic valve (1303a, 1303b, 1303c or 1303d) is respectively opened; the draught fan 1305 is started; the waste gas generated by each reactor during treatment is respectively introduced into the biological deodorization filter tower 1306 by the draught fan 1305, and is discharged after being absorbed by the bio-fillers in the biological deodorization filter tower 1306 and transformed to reach the standard.

### EMBODIMENT 2

An ecological pollution treatment method for pig farms based on combination of planting and breeding includes:

I. Source separation and water saving of enclosure: rainwater and sewage; drinking water and sewage are separated by a source separation water-saving enclosure 101; the rainwater and the remaining drinking water are discharged to ditches outside the enclosure instead of being mixed into feces and urine; water-saving enclosure flushing and mechanical manure scraping or manual dry manure cleaning are adopted to prevent the rainwater and the remaining drinking water from mixing into the feces and urine of pigs at source of the enclosure and minimize the feces and urine; the enclosure is cleaned with high-pressure spray nozzles or even high-pressure air; the pig feces cleaned by mechanical manure scraping or manual dry manure cleaning is piled up in a dry manure shed 301; then, the feces are conveyed to a high-temperature aerobic solid fermentation system 104 and an animal-derived feed factory 103 by the manure shed 301, respectively; and the feces and urine of the pigs and enclosure flushing water are conveyed to an adjusting tank 401.

II. Breeding of black soldier fly: a water content of the pig feces is adjusted to an appropriate range at first; then, the feces are inoculated with an appropriate amount of black soldier fly larvae of right age, to obtain a mixture of frass 201 and black soldier flies and ova 202 after growth and propagation for a period of time; the black soldier flies and ova 202 are dried and crushed to obtain a black soldier fly feed 203, which is mixed with the plant-derived feed 608 in a certain proportion and then is added with appropriate grains, trace elements and other ingredients to produce complete nutrition feed for breeding the pigs; and frass 201 is conveyed to the high-temperature aerobic solid fermentation reactors (304A, 304B, . . . , and 304M) by the conveying device for high-temperature aerobic fermentation to obtain an organic solid fertilizer 603.

III. High-temperature aerobic fermentation of feces:

(1) The electromagnetic valve 1107f is opened so that tap water is automatically replenished to the low-temperature thermal insulation water tank 1105; the electromagnetic valve 1107a is opened so that the low-temperature thermal insulation water tank 1105 automatically replenishes the water to the high-temperature thermal insulation water tank 1104 by utilizing a height difference; the electromagnetic valves (1107g, 1107b and 1107d) are opened; the circulating water pumps (1106a and 1106b) are started; the atmospheric pressure hot water boiler 1101 and the solar heating system 1103 are started; the water of the high-temperature thermal insulation water tank 1104 is conveyed to the atmospheric pressure hot water boiler 1101 and the solar heating system 1103 for heating; then, the electromagnetic valves (1107c and 1107e) are opened; the circulating water pumps (1106a and 1106b) are started; the hot water is conveyed to the high-temperature thermal insulation water tank 1104 for energy storage; the electromagnetic valve 1107i is opened; the electromagnetic valve (1107j, 1107k or 1107l) at the front end of the heating jacket or coil of the high-temperature aerobic solid fermentation reactor (304A, 304B or 304M) is opened; the hot water circulating pump 1106e is started; and the hot water is conveyed to heat up materials in the high-temperature aerobic solid fermentation reactor (304A, 304B or 304M).

(2) The solid feces and the auxiliary materials 302 in the dry manure shed 301 as well as the decomposing bacteria 303 are conveyed into the high-temperature aerobic solid fermentation reactor (304A, 304B or 304M) by the conveying, device; the water content of the mixture is controlled at 55-65%; and the detection and control system 109 simultaneously starts a driving device of the high-temperature aerobic solid fermentation reactor (304A, 304B or 304M) while adding the materials, thereby realizing feeding and stirring.

(3) After completing feeding, the detection and control system 109 controls the high-temperature aerobic solid fermentation reactor (304A, 304B or 304M) to stop stirring for 50 min, stir for 10 min, stop stirring for 50 min, and then stir for 10 min; a cycle of stop-stir-stop-stir-stop-stir is a timed stirring program; and meanwhile, the detection and control system 109 automatically starts the draught fan (1202a, 1202b or 1202m) to supply oxygen to fermented materials in the high-temperature aerobic solid fermentation reactor (304A, 304B or 304M) when the high-temperature aerobic solid fermentation reactor (304A, 304B or 304M) stirs. <1> The hot water enters the heating jacket or coil to rise the temperature of the materials in the high-temperature aerobic solid fermentation reactor (304A, 304B or 304M) when the detection and control system 109 detects that the temperature of the materials in the high-temperature aerobic solid fermentation reactor (304A, 304B or 304M) is lower than a set temperature 60° C. of the materials. <2> The timed stirring program is stopped and changed to a temperature-controlled stirring program when the temperature of the materials in the high-temperature aerobic solid fermentation reactor (304A, 304B or 304M) is greater than or equal to 70° C.; the draught fan (1202a, 1202b or 1202m) is started, the high-temperature aerobic solid fermentation reactor (304A, 304B or 304M) is driven to stir; the timed stirring program is not started until the temperature of the materials in the high-temperature aerobic solid fermentation reactor (304A, 304B or 304M) is lower than 70° C.; the temperature of the materials in the high-temperature aerobic solid fermentation reactor (304A, 304B or 304M) is maintained in 60-70° C.; and the timed stirring program and the temperature-controlled stirring program of the high-temperature aerobic solid fermentation reactor (304A, 304B or 304M) are adopted to establish an appropriate fermentation temperature and provide sufficient oxygen for the materials in the high-temperature aerobic solid fermentation reactor (304A, 304B or 304M) and establish an appropriate environment for high-temperature aerobic fermentation of feces solids.

(4) The high-temperature aerobic fermentation is completed in 24 h after feeding the materials; the detection and control system 109 controls the high-temperature solid fermentation reactor (304A, 304B or 304M) to stop, immediately feed 50% of materials after discharging 50% of the materials, and then immediately feed 50% of materials after discharging 50% of the materials every 24 h; similarly, a spiral discharge machine is started at first when discharging the materials; and meanwhile, the detection and control system 109 controls the high-temperature solid fermentation reactor (304A, 304B or 304M) to stir and guide the discharge.

(5) The materials discharged from the high-temperature aerobic solid fermentation reactor (304A, 304B or 304M) are conveyed to the aging chamber 305 by the conveying device; the materials are regularly turned or aerated in this period so that the materials are cooled and lose water until the materials are completely decomposed to prepare the organic fertilizer.

(6) The detection and control system 109 respectively detects the temperature of the materials in each high-temperature aerobic solid fermentation reactor (304A, 304B or 304M) when the high-temperature aerobic solid fermentation reactors (304A, 304B and 304M) simultaneously ferment, so that the temperature of the materials in each high-temperature aerobic solid fermentation reactor (304A, 304B or 304M) is maintained in 60-70° C.

(7) The detection and control system 109 detects and controls the water temperature in the high-temperature thermal insulation water tank 1104 to keep the water temperature constant in 70-85° C. <1> The atmospheric pressure hot water boiler 1101 is started when the temperature in the high-temperature thermal insulation water tank 1104 is lower than 70° C.; and the circulating pump 1106e at the output end of the solar heating system 1103 is started to convey the hot water to the high-temperature thermal insulation water tank 1104 when the temperature of the hot water in the heat collecting water tank of the solar heating system 1103 is greater than 70° C.; and <2> the atmospheric pressure hot water boiler is shut down when the temperature in the high-temperature thermal insulation water tank 1104 reaches 85° C.

IV. Medium-temperature anaerobic fermentation, of manure liquid:

(1) The feces and urine and the enclosure flushing water are conveyed into the adjusting tank 401, so that the liquid level of the manure liquid in the adjusting tank 401 keeps rising; the manure liquid naturally flows into the first medium-temperature anaerobic liquid fermentation reactor 402A along the connecting pipe due to the height difference when the liquid level is higher than the liquid outlet of the adjusting tank 401; the detection and control system 109 controls to open the electromagnetic valve 1107s at the front end of the heating coil of the first medium-temperature anaerobic liquid fermentation reactor 402A; the hot water enters the heating coil 504 of the first medium-temperature anaerobic liquid fermentation reactor 402A for circulation to rapidly rise the temperature of the materials in the first medium-temperature anaerobic liquid fermentation reactor 402A to 35° C.; and the materials start to perform the medium-temperature anaerobic fermentation reaction.

(2) The manure liquid naturally flows into the second medium-temperature anaerobic liquid fermentation reactor 402B along the connecting pipe due to the height difference when the liquid level of the liquid in the first medium-temperature anaerobic liquid fermentation reactor 402A is higher than the liquid outlet; the detection and control system 109 controls to open the electromagnetic valve 1107t at the front end of the heating coil of the second medium-temperature anaerobic liquid fermentation reactor 402B, so that the hot water enters the heating coil of the second medium-temperature anaerobic liquid fermentation reactor 402B for circulation to rapidly rise the temperature of the materials to 35° C., and the materials keep performing the medium-temperature anaerobic fermentation reaction.

(3) The manure liquid naturally flows out along the connecting pipe due to the height difference when the liquid level of the liquid in the second medium-temperature anaerobic liquid fermentation reactor 402B is higher than the liquid outlet, and so on, until the manure liquid naturally flows into the Nth medium-temperature anaerobic liquid fermentation reactor 402N; the detection and control system 109 controls to open the electromagnetic valve 1107u at the front end of the heating coil 504 of the Nth medium-temperature anaerobic liquid fermentation reactor 402N, so that the hot water enters the heating coil 504 of the Nth medium-temperature anaerobic liquid fermentation reactor 402N for circulation to rapidly rise the temperature of the materials to 35° C., and the materials keep performing the medium-temperature anaerobic fermentation reaction.

(4) Polarizers 513 in the medium-temperature anaerobic liquid fermentation reactors (402A, 402B, . . . , and 402N) are started in a time-division manner, to prevent the liquid surface of the liquid from “crusting” and slow down sedimentation of liquid sediments.

(5) The detection and control system 109 respectively detects and controls the temperature of the materials in the medium-temperature anaerobic liquid fermentation reactors (402A, 402B, . . . , and 402N) to keep the water temperature constant within a range of 35-50° C.; the manure liquid sequentially flows through N medium-temperature anaerobic liquid fermentation reactors (402A, 402B, . . . , and 402N); and fermentation liquid in the Nth medium-temperature anaerobic liquid fermentation reactor 402N naturally flows into the liquid outlet tank 403 along the pipe due to the height difference, to prepare the biogas fertilizer.

(5) The detection and control system 109 regularly starts the sludge pump 404 to control the liquid level of the liquid outlet tank 403 according to a set anaerobic fermentation time, and ensures the manure liquid to stay in the medium-temperature anaerobic liquid fermentation reactors (402A, 402B, . . . , and 402N) for more than 15 days; the biogas slurry in the liquid outlet tank 403 is pumped into the liquid storage tank 405 by the sludge pump 404 so that the liquid level of the liquid in the liquid outlet tank 403 is lowered after the time for medium-temperature anaerobic fermentation reaches 15 days; and the sludge pump 404 is turned off after the detection and control system 109 detects that the liquid level of the liquid outlet tank reaches a lower limit of the liquid level.

(6) The biogas generated by the medium-temperature anaerobic liquid fermentation reactors (402A, 402B, . . . , and 402N) is conveyed to the biogas pretreatment device 1112 through the biogas exhaust pipe 510 and the conveying pipe for treatment, and then is used as a combustion fuel of the steam generator 1102. The steam generator 1102 also uses electricity, diesel and biomass as supplementary fuels when the temperature is low in winter.

V. Cracking and breeding of sick and dead pigs and placentas:

(1) Cracking of sick and dead pigs and placentas:

<1> The forklift or other transfer equipment is used to place large sick and dead pigs into the net cage 1002; the net cage 1002 is pushed into the cracking and propagation reactors 701 by the conveying device; the forklift or other transfer equipment is used to place the small sick and dead pigs and placentas into the net cage 1002; the net cage 1002 is pushed into the cracking reactor (703A or 703B) by the conveying device; the cover sealing door is closed; and the liquid in the adjusting tank 401 is conveyed into the cracking and propagation reactors 701 and the cracking reactor (703A or 703B) by the conveying pumps so that the net cages 1002 are semi-immersed in the manure liquid.

<2> The electromagnetic valve 1107b on the water inlet pipe of the steam generator is opened; the hot water in the high-temperature thermal insulation water tank 1104 is pumped into the steam generator 1102; then, the steam generator 1102 is started; finally, the electromagnetic valve (1304e, 1304b or 1304c) at the front ends of the steam inlet pipes of the cracking and propagation reactors 701 and the cracking reactor (703A or 703B) is respectively opened; hot steam generated by the steam generator 1102 is respectively conveyed into the cracking and propagation reactors 701 and the cracking reactor (703A or 703B) so that the temperature and the pressure of the liquid in the cracking and propagation reactors 701 and the cracking reactor (703A or 703B) are increased; the exhaust valves of the cracking and propagation reactors 701 and the cracking reactor (703A or 703B) are respectively closed after exhausting cold air in the cracking and propagation reactors 701 and the cracking reactor (703A or 703B), so that the temperature and the pressure in the cracking and propagation reactors 701 and the cracking reactor (703A or 703B) respectively reach 130° C. and 0.25 Mpa; the sick and dead pigs and the placentas start to be cracked at high temperature and high pressure; and the detection and control system 109 detects and controls the temperature and the pressure in the cracking and propagation reactors 701 and the cracking reactor (703A or 703B) to keep them constant within 130-140° C. and 0.25-0.35 Mpa for more than 30 min, so that the sick and dead pigs are completely harmless, and are disintegrated and dissolved in the liquid to obtain the cracking liquid.

<3> The steam generator 1102 is turned off after high-temperature and high-pressure cracking is completed; the electromagnetic valve 1107y is opened; the electromagnetic valve (1107m, 1107n or 1107o) on circulating water inlet pipes of the cracking and propagation reactors 701 and the cracking reactor (703A or 703B) is opened; the electromagnetic valve 1107i is opened; the circulating water pump 1106e is started; the hot water enters the water jackets of the cracking and propagation reactors 701 and the cracking reactor (703A or 703B) for circulation to balance the. temperature of the cracking liquid with the temperature of the hot water of the high-temperature thermal insulation water tank 1104; then, the circulating water pump 1106e and the electromagnetic valve 1107i on the water outlet pipe of the high-temperature thermal insulation water tank 1104 are closed; the electromagnetic valve 1107y is closed; the circulating water pump 1106d and the electromagnetic valve 1107h on the water outlet pipe of the low-temperature thermal insulation water tank 1105 are started; cold water enters the water jackets of the cracking and propagation reactors 701 and the cracking reactor (703A or 703B) for circulation so that the cracking liquid is cooled to 25-35° C.; and the circulating water pump 1106d and the electromagnetic valve 1107h are closed.

(2) Breeding of feed insects with the cracking liquid

<1> The cracking liquid in the cracking and propagation reactors 701 and the cracking reactor (703A or 703B) is conveyed into the feed factory 103; the livestock and poultry feces are added into the cracking liquid and mixed uniformly; and a mixture is used as an insect feed for breeding insects to obtain insects and ova 202, which are used as the animal-derived feed 203, are mixed with a plant-derived feed 608 in a certain proportion and then are added with appropriate grains, trace elements and other ingredients to produce complete nutrition feed for breeding the pigs.

<2> The residual mixture of the cracking liquid and the livestock and poultry feces as well as frass are conveyed to the high-temperature aerobic solid fermentation reactors (304A, 304B and 304M) by the conveying device for high-temperature aerobic fermentation to obtain the organic solid fertilizer 603.

VI. Planting of feed crops:

(1) An appropriate amount of NPK fertilizer 602 is added into the biogas slurry to prepare the organic-inorganic compound liquid fertilizer 604 according to growth demands of Pennisetum purpureum Schumev.Taiwa.

(2) An appropriate amount of organic solid fertilizer 603 and organic-inorganic compound liquid fertilizer 604 are respectively applied according to the growth demands of the feed crops before and during planting of the Pennisetum purpureum Schumev.Taiwa according to the breeding stock of a pig farm and the supporting planting land 607 with the assimilative capacity of the Pennisetum purpureum Schumev.Taiwa; and the Pennisetum purpureum Schumev.Taiwa is harvested and conveyed to the plant-derived feed factory 103 and is processed to prepare the plant-derived feed 608, which is mixed with the black soldier fly feed 203 in a certain proportion and then is added with appropriate grains, trace elements and other ingredients to produce the complete nutrition feed for breeding the pigs.

VII. Waste gas treatment:

(1) Treatment for odor fermented by the high-temperature aerobic solid fermentation system 104: the electromagnetic valve (1205a, 1205b, . . . , or 1205m) on the exhaust pipe of the high-temperature aerobic solid fermentation reactor (304A, 304B, . . . , or 304M) is respectively opened; the odor generated during fermentation is respectively introduced into the biological deodorization filter tower 1203 by the draught fan (1202a, 1202b, . . . , or 1202m) after respectively exchanging heat by the heat exchange condenser (1201a, 1201b, . . . , or 1201m) and is discharged after being absorbed by bio-fillers in the biological deodorization filter tower 1203 and transformed to reach a standard; and meanwhile, the fresh air heated by the heat exchange condenser (1201a, 1201b, . . . , or 1201m) is respectively introduced into the high-temperature aerobic solid fermentation reactor (304A, 304B, . . . , or 304M).

(2) Treatment for waste gases fermented by the cracking and propagation system 106: the electromagnetic valve (1303a, 1303b, 1303c or 1303d) is respectively opened; the draught fan 1305 is started; and the waste gas generated by each reactor during treatment is respectively introduced into the biological deodorization filter tower 1306 by the draught fan 1305, and is discharged after being absorbed by the bio-fillers in the biological deodorization filter tower 1306 and transformed to reach the standard.

### EMBODIMENT 3

An ecological pollution treatment method for chicken farms based on combination of planting and breeding includes:

I. Source separation and water saving of enclosure: rainwater and sewage, drinking water and sewage are separated by a source separation water-saving enclosure 101; the rainwater and the remaining drinking water are discharged to ditches outside the enclosure instead of being mixed into feces, thereby minimizing the feces; chicken feces cleaned by mechanical manure scraping or manual dry manure cleaning is piled up in the dry manure shed 301; and then, the feces is conveyed to the high-temperature aerobic solid fermentation system 104 and the animal-derived feed factory 103 by the manure shed 301, respectively.

II. Breeding of black soldier fly: a water content of the pig feces is adjusted to an appropriate range at first; then, the feces are inoculated with an appropriate amount of black soldier fly larvae of right age, to obtain a mixture of frass 201 and black soldier flies and ova 202 after growth and propagation for a period of time; the black soldier flies and ova 202 are dried and crushed to obtain a black soldier fly feed 203, which is mixed with the plant-derived feed 608 in a certain proportion and then is added with appropriate grains, trace elements and other ingredients to produce complete nutrition feed for breeding chickens; and frass 201 is conveyed to the high-temperature aerobic solid fermentation reactors (304A, 304B, . . . , and 304M by the conveying device for high-temperature aerobic fermentation to obtain the organic solid fertilizer 603.

III. High-temperature aerobic fermentation of feces:

(1) The electromagnetic valve 1107f is opened so that tap water is automatically replenished to the low-temperature thermal insulation water tank 1105; the electromagnetic valve 1107a is opened so that the low-temperature thermal insulation water tank 1105 automatically replenishes the water to the high-temperature thermal insulation water tank 1104 by utilizing a height difference; the electromagnetic valves (1107g, 1107b and 1107d) are opened; the atmospheric pressure hot water boiler 1101 and the solar heating system 1103 are started; the water of the high-temperature thermal insulation water tank 1104 is conveyed to the atmospheric pressure hot water boiler 1101 and the solar heating system 1103 for heating; then, the electromagnetic valves (1107c and 1107e) are opened; the circulating water pumps (1106a and 1106b) are started; the hot water is conveyed to the high-temperature thermal insulation water tank 1104 for energy storage; the electromagnetic valve 1107i is opened; the electromagnetic valve (1107j, 1107k or 1107l) at the front end of the heating jacket or coil of the high-temperature aerobic solid fermentation reactor (304A, 304B or 304M) is opened, the hot water circulating pump 1106e is started; and the hot water is conveyed to heat up materials in the high-temperature aerobic solid fermentation reactor (304A, 304B or 304M).

(2) The solid feces and the auxiliary materials 302 in the dry manure shed 301 as well as the decomposing bacteria 303 are conveyed into the high-temperature aerobic solid fermentation reactor (304A, 304B or 304M) by the conveying device; the water content of the mixture is controlled at 55-65%; and the detection and control system 109 simultaneously starts a driving device of the high-temperature aerobic solid fermentation reactor (304A, 304B or 304M) while adding the materials, thereby realizing feeding and stirring.

(3) After completing feeding, the detection. and control system 109 controls the high-temperature aerobic solid fermentation reactor (304A, 304B or 304M) to stop stirring for 50 min, stir for 10 min, stop stirring for 50 min, and then stir for 10 min; a cycle of stop-stir-stop-stir-stop-stir is a timed stirring program; and meanwhile, the detection and control system 109 automatically starts the draught fan (1202a, 1202b or 1202m) to supply oxygen to fermented materials in the high-temperature aerobic solid fermentation reactor (304A, 304B or 304M) when the high-temperature aerobic solid fermentation reactor (304A, 304B or 304M) stirs. <1> The hot water enters the heating jacket or coil to rise the temperature of the materials in the high-temperature aerobic solid fermentation reactor (304A, 304B or 304M) when the detection and control system 109 detects that the temperature of the materials in the high-temperature aerobic solid fermentation reactor (304A, 304B or 304M) is lower than a set temperature 60° C. of the materials. <2> The timed stirring program is stopped and changed to a temperature-controlled stirring program when the temperature of the materials in the high-temperature aerobic solid fermentation reactor (304A, 304B or 304M) is greater than or equal to 70° C.; the draught fan (1202a, 1202b or 1202m) is started, the high-temperature aerobic solid fermentation reactor (304A, 304B or 304M) is driven to stir; the timed stirring program is not started until the temperature of the materials in the high-temperature aerobic solid fermentation reactor (304A, 304B or 304M) is lower than 70° C.; the temperature of the materials in the high-temperature aerobic solid fermentation reactor (304A, 304B or 304M) is maintained in 60-70° C.; and the timed stirring program and the temperature-controlled stirring program of the high-temperature aerobic solid fermentation reactor (304A, 304B or 304M) are adopted to establish an appropriate fermentation temperature and provide sufficient oxygen for the materials in the high-temperature aerobic solid fermentation reactor (304A, 304B or 304M) and establish an appropriate environment for high-temperature aerobic fermentation of feces solids.

(4) The high-temperature aerobic fermentation is completed in 24 h after feeding the materials; the detection and control system 109 controls the high-temperature solid fermentation reactor (304A, 304B or 304M) to stop, immediately feed 50% of materials after discharging 50% of the materials, and then immediately feed 50% of materials after discharging 50% of the materials every 24 h; similarly, a spiral discharge machine is started at first when discharging the materials; and meanwhile, the detection and control system 109 controls the high-temperature solid fermentation reactor (304A, 304B or 304M) to stir and guide the discharge.

(5) The materials discharged from the high-temperature aerobic solid fermentation reactor (304A, 304B or 304M) are conveyed to the aging chamber 305 by the conveying device; and the materials are regularly turned or aerated in this period so that the materials are cooled and lose water until the materials are completely decomposed to prepare an organic fertilizer.

(6) The detection and control system 109 respectively detects the temperature of the materials in each high-temperature aerobic solid fermentation reactor (304A, 304B or 304M) when the high-temperature aerobic solid fermentation reactors (304A, 304B and 304M) simultaneously ferment, so that the temperature Of the materials in each high-temperature aerobic solid fermentation reactor (304A, 304B or 304M) is maintained in 60-70° C.

(7) The detection and control system 109 detects and controls the water temperature in the high-temperature thermal insulation water tank 1104 to keep the water temperature constant in 70-85° C. <1> The atmospheric pressure hot water boiler 1101 is started when the temperature in the high-temperature thermal insulation water tank 1104 is lower than 70° C.; and the circulating pump 1106e at the output end of the solar heating system 1103 is started to convey the hot water to the high-temperature thermal insulation water tank 1104 when the temperature of the hot water in the heat collecting water tank of the solar heating system 1103 is greater than 70° C.; and <2> the atmospheric pressure hot water boiler is shut down when the temperature in the high-temperature thermal insulation water tank 1104 reaches 85° C.

4. Planting of feed crops:

(1) An appropriate amount of NPK fertilizer 602 is added into the organic solid fertilizer to prepare an organic-inorganic compound solid fertilizer according to growth demands of corns.

(2) An appropriate amount of organic solid fertilizer 603 and organic-inorganic compound fertilizer are respectively applied according to nutrient requirements of the corns in different growth periods before and during planting of the corns according to the breeding stock of a chicken farm and the assimilative capacity supporting planting land 607 of the corns; the harvested corns are conveyed to the plant-derived feed factory 103 and is processed to prepare the plant-derived feed 608, which is mixed with the animal-derived feed in a certain proportion and then is added with appropriate grains, trace elements and other ingredients to produce the complete nutrition feed for breeding the chickens; while corn straws are used as the auxiliary materials to be conveyed into the high-temperature aerobic solid fermentation reactor and mixed with chicken feces for high-temperature fermentation to prepare the organic solid fertilizer.

5. Waste gas treatment:

The electromagnetic valve (1205a, 1205b, . . . , or 1205m) on the exhaust pipe of the high-temperature aerobic solid fermentation reactor (304A, 304B, . . . , or 304M) is respectively opened; the odor generated during fermentation is respectively introduced into the biological deodorization filter tower 1203 by the draught fan (1202a, 1202b, . . . , or 1202m) after respectively exchanging heat by the .heat exchange condenser (1201a, 1201b, . . . , or 1201m) and is discharged after being absorbed by bio-fillers in the biological deodorization filter tower 1203 and transformed to reach a standard; and meanwhile, the fresh air heated by the heat exchange condenser (1201a, 1201b, . . . , or 1201m) is respectively introduced into the high-temperature aerobic solid fermentation reactor (304A, 304B, . . . , or 304M).

### EMBODIMENT 4

An ecological pollution treatment method for cattle farms based on combination of planting and breeding includes:

I. Source separation and water saving of enclosure: rainwater and sewage, drinking water and sewage are separated by the source separation water-saving enclosure 101; the rainwater and the remaining drinking water are discharged to the ditches outside the enclosure instead of being mixed into feces and urine; water-saving enclosure flushing and mechanical manure scraping or manual dry manure cleaning are adopted to prevent the rainwater and the remaining drinking water from mixing into the feces and urine of cattle at source of the enclosure and minimize the feces and urine; the enclosure is cleaned with high-pressure spray nozzles or even high-pressure air; the cattle feces cleaned by mechanical manure scraping or manual dry manure cleaning are piled up in the dry manure shed 301; then, the feces is conveyed to the high-temperature aerobic solid fermentation system 104 by the manure shed 301, respectively; and the feces and urine of the cattle and enclosure flushing water are conveyed to the adjusting tank 401.

II. High-temperature aerobic fermentation of feces:

(1) The electromagnetic valve 1107f is opened so that tap water is automatically replenished to the low-temperature thermal insulation water tank 1105; the electromagnetic valve 1107a is opened so that the low-temperature thermal insulation water tank 1105 automatically replenishes the water to the high-temperature thermal insulation water tank 1104 by utilizing the height difference; the electromagnetic valves (1107g, 1107b and 1107d) are opened; the circulating water pumps (1106a and 1106b) are started; the atmospheric pressure hot water boiler 1101 and the solar heating system 1103 are started; the water of the high-temperature thermal insulation water tank 1104 is conveyed to the atmospheric pressure hot water boiler 1101 and the solar heating system 1103 for heating; then, the electromagnetic valves (1107c and 1107e) are opened; the circulating water pumps (1106a and 1106b) are started; the hot water is conveyed to the high-temperature thermal insulation water tank 1104 for energy storage; the electromagnetic valve 1107i is opened; the electromagnetic valve (1107j, 1107k or 1107l) at the front end of the heating jacket or coil of the high-temperature aerobic solid fermentation reactor (304A, 304B or 304M) is opened; the hot water circulating pump 1106e is started; and the hot water is conveyed to heat up materials in the high-temperature aerobic solid fermentation reactor (304A, 304B or 304M).

(2) The solid feces and the auxiliary materials 302 in the dry manure shed 301 as well as the decomposing bacteria 303 are conveyed into the high-temperature aerobic solid fermentation reactor (304A, 304B or 304M) by the conveying device; the water content of the mixture is controlled at 55-65%; and the detection and control system 109 simultaneously starts the driving device of the high-temperature aerobic solid fermentation reactor (304A, 304B or 304M) while adding the materials, thereby realizing feeding and stirring.

(3) After completing feeding, the detection and control system 109 controls the high-temperature aerobic solid fermentation reactor (304A, 304B or 304M) to stop stirring for 50 min, stir for 10 min, stop stiffing for 50 min, and then stir for 10 min; a cycle of stop-stir-stop-stir-stop-stir is a timed stirring program; and meanwhile, the detection and control system 109 automatically starts the draught fan (1202a, 1202b or 1202m) to supply oxygen to fermented materials in the high-temperature aerobic solid fermentation reactor (304A, 304B or 304M) when the high-temperature aerobic solid fermentation reactor (304A, 304B or 304M) stirs. <1> The hot water enters the heating jacket or coil to rise the temperature of the materials in the high-temperature aerobic solid fermentation reactor (304A, 304B or 304M) when the detection and control system 109 detects that the temperature of the materials in the high-temperature aerobic solid fermentation reactor (304A, 304B or 304M) is lower than a set temperature 60° C. of the materials. <2> The timed stirring program is stopped and changed to a temperature-controlled stirring program when the temperature of the materials in the high-temperature aerobic solid fermentation reactor (304A, 304B or 304M) is greater than or equal to 70° C.; the draught fan (1202a, 1202b or 1202m) is started, the high-temperature aerobic solid fermentation reactor (304A, 304B or 304M) is driven to stir; the timed stirring program. is not started until the temperature of the materials in the high-temperature aerobic solid fermentation reactor (304A, 304B or 304M) is lower than 70° C.; the temperature of the materials in the high-temperature aerobic solid fermentation reactor (304A, 304B or 304M) is maintained in 60-70° C.; and the timed stirring, program and the temperature-controlled stirring program of the high-temperature aerobic solid fermentation reactor (304A, 304B or 304M) are adopted to establish an appropriate fermentation temperature and provide sufficient oxygen for the materials in the high-temperature aerobic solid fermentation reactor (304A, 304B or 304M) and establish an appropriate environment for high-temperature aerobic fermentation of feces solids.

(4) The high-temperature aerobic fermentation is completed in 24 h after feeding the materials; the detection and control system 109 controls the high-temperature solid fermentation reactor (304A, 304B or 304M) to stop, immediately feed 50% of materials after discharging 50% of the materials, and then immediately feed 50% of materials after discharging 50% of the materials every 24 h; similarly, the spiral discharge machine is started at first when discharging the materials; and meanwhile, the detection and control system 109 controls the high-temperature solid fermentation reactor (304A, 304B or 304M) to stir and guide the discharge.

(5) The materials discharged from the high-temperature aerobic solid fermentation reactor (304A, 304B or 304M) are conveyed to the aging chamber 305 by the conveying device; and the materials are regularly turned or aerated in this period so that the materials are cooled and lose water until the materials are completely decomposed to prepare the organic fertilizer.

(6) The detection and control system 109 respectively detects the temperature of the materials in each high-temperature aerobic solid fermentation reactor (304A, 304B or 304M) when the high-temperature aerobic solid fermentation reactors (304A, 304B and 304M) simultaneously ferment, so that the temperature of the materials in each high-temperature aerobic solid fermentation reactor (304A, 304B or 304M) is maintained in 60-70° C.

(7) The detection and control system 109 detects and controls the water temperature in the high-temperature thermal insulation water tank 1104 to keep the water temperature constant in 70-85° C. <1> The atmospheric pressure hot water boiler 1101 is started when the temperature in the high-temperature thermal insulation water tank 1104 is lower than 70° C.; and the circulating pump 1106e at the output end of the solar heating system 1103 is started to convey the hot water to the high-temperature thermal insulation water tank 1104 when the temperature of the hot water in the heat collecting water tank of the solar heating system 1103 is greater than 70° C.; and <2> the atmospheric pressure hot water boiler is shut down when the temperature in the high-temperature thermal insulation water tank 1104 reaches 85° C.

III. Medium-temperature anaerobic fermentation of manure liquid:

(1) The feces and urine and the enclosure flushing water are conveyed into the adjusting tank 401, so that a liquid level of the manure liquid in the adjusting tank 401 keeps rising; the manure liquid naturally flows into the first medium-temperature anaerobic liquid fermentation reactor 402A along the connecting pipe due to the height difference when the liquid level is higher than the liquid outlet of the adjusting tank 401; the detection and control system 109 controls to open the electromagnetic valve 1107s at the front end of the heating coil of the first medium-temperature anaerobic liquid fermentation reactor 402A; the hot water enters the heating coil 504 of the first medium-temperature anaerobic liquid fermentation reactor 402A for circulation to rapidly rise the temperature of the materials in the first medium-temperature anaerobic liquid fermentation reactor 402A to 35° C.; and the materials start to perform the medium-temperature anaerobic fermentation reaction.

(2) The manure liquid naturally flows into the second medium-temperature anaerobic liquid fermentation reactor 402B along the connecting pipe due to the height difference when the liquid level of the liquid in the first medium-temperature anaerobic liquid fermentation reactor 402A is higher than the liquid outlet; the detection and control system 109 controls to open the electromagnetic valve 1107t at the front end of the heating coil of the second medium-temperature anaerobic liquid fermentation reactor 402B, so that the hot water enters the heating coil of the. second medium-temperature anaerobic liquid fermentation reactor 402B for circulation to rapidly rise the temperature of the materials to 35° C., and the materials keep performing the medium-temperature anaerobic fermentation reaction.

(3) The manure liquid naturally flows out along the connecting pipe due to the height difference when: the liquid level of the liquid in the second medium-temperature anaerobic liquid fermentation reactor 402B is higher than the liquid outlet, and so on until the manure liquid naturally flows into the Nth medium-temperature anaerobic liquid fermentation reactor 402N; the detection and control system 109 controls to open the electromagnetic valve 1107u at the front end of the heating coil 504 of the Nth medium-temperature anaerobic liquid fermentation reactor 402N, so that the hot water enters the heating coil 504 of the Nth medium-temperature anaerobic liquid fermentation reactor 402N for circulation to rapidly rise the temperature of the materials to 35° C., and the materials keep performing the medium-temperature anaerobic fermentation reaction.

(4) Polarizers 513 in the medium-temperature anaerobic liquid fermentation reactors (402A, 402B, . . . , and 402N) are started in a time-division manner, to prevent the liquid surface. of the liquid from “crusting” and slow down sedimentation of liquid sediments.

(5) The detection and control system 109 respectively detects and controls the temperature of the materials in the medium-temperature anaerobic liquid fermentation reactors (402A, 402B, . . . , and 402N) to keep the water temperature constant within a range of 35-50° C.; the manure liquid sequentially flows through N medium-temperature anaerobic liquid fermentation reactors (402A, 402B, . . . , and 402N); and fermentation liquid in the Nth medium-temperature anaerobic liquid fermentation reactor 402N naturally flows into the liquid outlet tank 403 along the pipe due to the height difference, to prepare the biogas fertilizer.

(6) The detection and control system 109 regularly starts the sludge pump 404 to control the liquid level of the liquid outlet tank 403 according to a set anaerobic fermentation time, and ensures the manure liquid to stay in the medium-temperature anaerobic liquid fermentation reactors (402A, 402B, . . . , and 402N) for more than 15 days; the biogas slurry in the liquid outlet tank 403 is pumped into the liquid storage tank 405 by the sludge pump 404 so that the liquid level of the liquid in the liquid outlet tank 403 is lowered after the time for medium-temperature anaerobic fermentation reaches 15 days; and the sludge pump 404 is turned off after the detection and control system 109 detects that the liquid level of the liquid outlet tank reaches a lower limit of the liquid level.

(7) The biogas generated by the medium-temperature anaerobic liquid fermentation reactors (402A, 402B, . . . , and 402N) is conveyed to the biogas pretreatment device 1112 through the biogas exhaust pipe 510 and the conveying pipe for treatment, and then is used as the combustion fuel of the steam generator 1102. The steam generator 1102 also uses electricity, diesel and biomass as supplementary fuels when the temperature is low in winter.

IV. Cracking and propagation of sick and dead cattle and placentas:

(1) Cracking of sick and dead cattle and placentas:

<1> The forklift or other transfer equipment is used to place large sick and dead cattle into the net cage 1002; the net cage 1002 is pushed into the cracking and propagation reactors 701 by the conveying device; the forklift or other transfer equipment is used to place the small sick and dead calves and placentas into the net cage 1002; the net cage 1002 is pushed into the cracking reactor (703A or 703B) by the conveying device; the cover sealing door is closed; and the liquid in the adjusting tank 401 is conveyed into the cracking and propagation reactors 701 and the cracking reactor (703A or 703B) by the conveying pumps so that the net cages 1002 are semi-immersed in the manure liquid.

<2> The electromagnetic valve 1107b on the water inlet pipe of the steam generator is opened; the hot water in the high-temperature thermal insulation water tank 1104 is pumped into the steam generator 1102; then, the steam generator 1102 is started; finally, the electromagnetic valve (1304e, 1304b or 1304c) at the front ends of the steam inlet pipes of the cracking and propagation reactors 701 and the cracking reactor (703A or 703B) is respectively opened; hot steam generated by the steam generator 1102 is respectively conveyed into the cracking and propagation reactors 701 and the cracking reactor (703A or 703B) so that the temperature and the pressure of the liquid in the cracking and propagation reactors 701 and the cracking reactor (703A or 703B) are increased; the exhaust valves of the cracking and propagation reactors 701 and the cracking reactor (703A or 703B) are respectively closed after exhausting cold air in the cracking and propagation reactors 701 and the cracking reactor (703A or 703B), so that the temperature and the pressure in the cracking and propagation reactors 701 and the cracking reactor (703A or 703B) respectively reach 130° C. and 0.25 Mpa; the sick and dead pigs and the placentas start to be cracked at high temperature and high pressure; and the detection and control system, 109 detects and controls the temperature and the pressure in the cracking and propagation reactors 701 and the cracking reactor (703A or 703B) to keep. them constant within 130-140° C. and 0.25-0.35 Mpa for more than 30 min, so that the sick and dead pigs are completely harmless, and are disintegrated and dissolved in the liquid to obtain the cracking liquid.

<3> The steam generator 1102 is turned off after high-temperature and high-pressure cracking is completed; the electromagnetic valve 1107y is opened; the electromagnetic valve (1107m, 1107n or 1107o) on steam inlet pipes of the cracking and propagation reactors 701 and the cracking reactor (703A or 703B) is opened; the electromagnetic valve 1107i is opened; the circulating water pump 1106e is started; the hot water enters the water jackets of the cracking and propagation reactors 701 and the cracking reactor (703A or 703B) for circulation to balance the temperature of the cracking liquid with the temperature of the hot water of the high-temperature thermal insulation water tank 1104; then, the circulating water pump 1106e and the electromagnetic valve 1107i on the water outlet pipe of the high-temperature thermal insulation water tank 1104 are closed; the electromagnetic valve 1107y is closed; the circulating water pump 1106d and the electromagnetic valve 1107h on the water outlet pipe of the low-temperature thermal insulation water tank 1105 are started; cold water enters the water jackets of the cracking and propagation reactors 701 and the cracking reactor (703A or 703B) for circulation so that the cracking liquid is cooled to 25-35° C.; and the circulating water pump 1106d and the electromagnetic valve 1107h are closed.

<4> The electromagnetic valve on the discharge pipe of the cracking reactor (703A or 703B) is opened; and the cracking liquid in the cracking reactor (703A or 703B) is conveyed into the propagation reactor 704.

(2) Propagation of the cracking liquid

<1> The pre-cultured microbial seed liquid is respectively conveyed into the cracking and propagation reactor 701 and the propagation reactor 704 through the feed pipe 812 of the cracking and propagation reactor 701 and the feed port 905 of the propagation reactor 704; the aeration fan 1301 is started; the detection and control system 109 respectively controls opening or closing of the electromagnetic valve 1304d and the electromagnetic valve 1304e; sterile air filtered by the air filter 1302 is respectively conveyed into the cracking and propagation reactor 701 and the propagation reactor 704; meanwhile, the detection and control system 109 detects and controls the temperature in the cracking and propagation reactor 701 and the propagation reactor 704 to respectively keep the temperature in 25-35° C.; and a detection and control method includes: 1) the detection and control system 109 controls to turn on the circulating water pump 1106e on the water outlet pipe of the high-temperature thermal insulation water tank 1104, the electromagnetic valve 1107i is opened, and the electromagnetic valve (1107m or 1107p) is opened when the detection and control system 109 detects that the temperature in the cracking and propagation reactor 701 or the propagation reactor 704 is lower than 25° C.; the circulating water pump 1106e is stopped, the electromagnetic valve 1107i is closed, and the electromagnetic valve (1107m or 1107p) is closed when the culture solution in the cracking and propagation reactor 701 and the propagation reactor 704 is heated up to 35° C.; 2) the detection and control system 109 controls to start the aeration fan 1301, and the electromagnetic valve (1304d or 1304e) is opened to aerate the cracking and propagation reactor 701 or the propagation reactor 704 when the detection and control system 109 detects that the temperature in the cracking and propagation reactor 701 or the propagation reactor 704 exceeds 35° C.; the aeration fan 1301 is turned off, and the electromagnetic valve (1304d or 1304e) is closed when the materials in the cracking and propagation reactor 701 or the propagation reactor 704 is cooled to 25-35° C.

<2> A propagation process is completed when the concentration of a culture bacteria solution reaches requirements after the cracking liquid is cultured for 3 days; the discharge pipe 809 of the cracking and propagation reactor 701 is respectively opened to exchange heat with the discharge pipe 910 at the bottom of the propagation reactor 704; the culture solution is discharged and stored in the liquid storage tank, and then is separated by an oil-water separator to obtain the microbial culture solution 702 and grease 705; and the grease 705 is used as an industrial raw material.

<3> The detection and control system 109 respectively detects and controls the temperature and the pressure of the materials in each reactor according to different cracking and propagation stages when a plurality of cracking and propagation reactors 701, the cracking reactors (703A and 703B) and the propagation reactor 704 react at the same time, so that the temperature and the pressure in each reactor are maintained within the set ranges.

VI. Planting of feed crops:

(1) The microbial culture solution 702 is sprayed to the aged organic solid. fertilizer 603 in a certain proportion, and are stirred uniformly to obtain a bio-organic fertilizer 606; the microbial culture solution 702 is added into the biogas slurry 601 in a certain proportion to obtain the microbial liquid fertilizer 605; and an appropriate amount of NPK fertilizer 602 is added into the biogas slurry to prepare an organic-inorganic compound liquid fertilizer 604 according to growth demands of Pennisetum hydridum.

(2) An appropriate amount of organic solid fertilizer 603, bio-organic fertilizer 606, microbial liquid fertilizer 605 and organic-inorganic compound liquid fertilizer 604 are respectively applied according to the growth demands of the feed crops before and during planting of the Pennisetum hydridum according to the breeding stock of a cattle farm and the supporting planting land 607 with the assimilative capacity of the Pennisetum hydridum; and the Pennisetum hydridum is harvested and conveyed to the plant-derived feed factory 103 and is processed to prepare the plant-derived feed 608, which is added with appropriate grains, trace elements and other ingredients to produce the complete nutrition feed for breeding the cattle.

VII. Waste gas treatment:

(1) Treatment for odor fermented by the high-temperature aerobic solid fermentation system 104: the electromagnetic valve (1205a, 1205b, or 1205m) on the exhaust pipe of the high-temperature aerobic solid fermentation reactor (304A, 304B, . . . , or 304M is respectively opened; the odor generated during fermentation is respectively introduced into the biological deodorization filter tower 1203 by the draught fan (1202a, 1202b, . . . , or 12021m) after respectively exchanging heat by the heat exchange condenser (1201a, 1201b, . . . , or 1201m) and is discharged after being absorbed by bio-fillers in the biological deodorization filter tower 1203 and transformed to reach a standard; and meanwhile, the fresh air heated by the heat exchange condenser (1201a, 1201b, . . . , or 1201m) is respectively introduced into the high-temperature aerobic solid fermentation reactor (304A, 304B, . . . , or 304M).

(2) Treatment for waste gases fermented by the cracking and propagation system 106: the electromagnetic valve (1303a, 1303b, 1303c or 1303d) is respectively opened; the draught fan 1305 is started; and the waste gas generated by each reactor during treatment is respectively introduced into the biological deodorization filter tower 1306 by the draught fan 1305, and is discharged after being absorbed by the bio-fillers in the biological deodorization filter tower 1306 and transformed to reach the standard.

We claim:

1. An ecological pollution treatment system for livestock and poultry farms based on combination of planting and breeding, comprising a source separation water-saving enclosure, a pollution treatment factory, a feed factory and a supporting planting land, wherein the pollution treatment factory and the feed factory are managed by professional teams for specialized disposal of feces of livestock and poultry; the pollution treatment factory is specialized in transforming and preparing manure of livestock and poultry into fertilizers needed to improve soil by utilizing a supporting pollution treatment workshop at the source; the feed factory is specialized in transforming and preparing the livestock and poultry manure into animal-derived feed and plant-derived feed by utilizing the supporting planting land and feed production workshop at the source; the pollution treatment factory is composed of a high-temperature aerobic solid fermentation system, a medium-temperature anaerobic liquid fermentation system, a cracking and propagation system, a heating and heat balancing system, a waste gas treatment system and a detection and control system; the source separation water-saving enclosure is a water-saving enclosure configured to separate rainwater and sewage, drinking water and sewage, discharge the rainwater and the remaining drinking water to outdoor ditches instead of mixing into the manure, prevent the rainwater and the remaining drinking water from mixing into the manure at the source of the enclosure by means of water-saving enclosure flushing and mechanical manure scraping or manual dry manure cleaning to minimize the manure, and flush the enclosure with high-pressure spray nozzles or even high-pressure air when slaughtering or transferring the livestock and poultry; the manure cleaned by mechanical manure scraping or manual dry manure cleaning is piled up in a dry manure shed; then, the manure is conveyed to the high-temperature aerobic solid fermentation system and the feed factory by the manure shed, respectively; frass generated in the feed factory is conveyed to the high-temperature aerobic solid fermentation system, while manure liquid and enclosure flushing water are conveyed to an adjusting tank; the adjusting tank is connected with a feed port of the medium-temperature anaerobic liquid fermentation system and a liquid inlet of the cracking and propagation system; an exhaust port of fermentation odor generated by the high-temperature aerobic solid fermentation system and an exhaust port of fermentation odor generated by the cracking and propagation are connected with the waste gas treatment system by exhaust pipes; the heating and heat balancing system is respectively connected with a heating jacket or coil of the high-temperature aerobic solid fermentation system, heating coils of the medium-temperature anaerobic liquid fermentation system as well as a water jacket and a coil of the cracking and propagation system by pipes; and sensors of the detection and control system are arranged in the above systems, to set, detect and control key parameters.

2. The ecological pollution treatment system for livestock and poultry farms based on combination of planting and breeding according to claim 1, wherein the feed factory is managed by the professional teams for specialized disposal of livestock manure and sewage, and is specialized in blending the livestock and poultry manure as well as cracking liquid of the dead livestock and poultry and placentas to prepare livestock and poultry manure for feeding insects to produce insects and ova and serving as the animal-derived feed while producing the plant-derived feed by using the livestock and poultry manure to plant grains and pastures on the supporting planting land at source; the planted grains and pastures comprise one or more of Pennisetum hydridum, Pennisetum purpureum, Alfalfa, Alternanthera, Lolium perenne, Chinese pennisetum, Broussonetia papyrifera, corns and soybeans; the bred insects comprise one or more of black soldier fly and earthworm; and the feed factory is specialized in adding appropriate grains and trace elements into the obtained plant-derived feed and animal-derived feed to produce complete nutrition feed for breeding the livestock and poultry.

3. The ecological pollution treatment system for livestock and poultry farms based on combination of planting and breeding according to claim 1, wherein the high-temperature aerobic solid fermentation system comprises M high-temperature aerobic solid fermentation reactors; M is a positive integer; the dry manure shed is respectively communicated with the feed port of each high-temperature aerobic solid fermentation reactor by the conveying device; and an aging chamber communicated with each high-temperature aerobic solid fermentation reactor is arranged at a discharge port of each high-temperature aerobic solid fermentation reactor.

4. The ecological pollution treatment system for livestock and poultry farms based on combination of planting and breeding according to claim 1, wherein the medium-temperature anaerobic liquid fermentation system comprises the adjusting tank, N medium-temperature anaerobic liquid fermentation reactors, a liquid outlet tank, a sludge pump and a liquid storage tank communicated in sequence, wherein N is a positive integer.

5. The ecological pollution treatment system for livestock and poultry farms based on combination of planting and breeding according to claim 4, wherein the medium-temperature anaerobic liquid fermentation reactors comprise soft anaerobic fermentation bags; a liquid outlet of the adjusting tank is connected with the feed port of a first soft anaerobic fermentation bag through a pipe; the discharge port of the soft anaerobic fermentation bag is connected with the feed port of a second soft anaerobic fermentation bag through the pipe, and so on, until the discharge port is connected to the feed port of an Nth soft anaerobic fermentation bag, and the discharge port thereof is connected with the liquid outlet tank through the pipe; then, the liquid outlet tank is connected with the liquid storage tank by the sludge pump; the medium-temperature anaerobic liquid fermentation reactors also comprise reaction tanks having rectangular bottoms; each reaction tank is arranged on an inclined surface with an inclination angle of 0.3-1% in a length direction of the reaction tank; a drainage ditch is formed around the inclined surface; the drainage ditch is connected to a water collecting well at the lower ground through the pipe; water collected at the bottoms of the reaction tanks is converged to the water collecting well through the drainage ditch for drainage; thermal insulation layers are arranged around the bottoms of the reaction tanks and the inclined surface at the bottoms of the tanks; the thermal insulation layers are made of thermal insulation materials; a heat radiation plate is arranged on the surface of the thermal insulation layer at the bottoms of the tanks; the heating coils are uniformly fixed on the heat radiation plate; the heating coils are covered with the soft anaerobic fermentation bags; the feed port is formed in a higher side of each soft anaerobic fermentation bag, and the discharge port is formed in a lower side of the same in the length direction; a polarizer is arranged in the middle of each rectangular reactor in the length direction; biogas pipes are arranged at the tops of the soft anaerobic fermentation bags; pressure sensors are arranged on the pipes; and the soft anaerobic fermentation bags are further covered with the thermal insulation layers and waterproof covers.

6. The ecological pollution treatment system for livestock and poultry farms based on combination of planting and breeding according to claim 4, wherein in the medium-temperature anaerobic liquid fermentation reactors connected in series with each other, the tank bottom at the feed port of a latter medium-temperature anaerobic liquid fermentation reactor is more than 0.2 in lower than that of the discharge port of a previous medium-temperature anaerobic liquid fermentation reactor to prevent sediments in the subsequent reactor from flowing back to the previous reactor; and for each medium-temperature anaerobic liquid fermentation reactor, the tank bottom at a feed port side is higher than that at a discharge side; and a range of inclination is 0.3-1% to reduce the number of sludge cleaning times of the medium-temperature anaerobic liquid fermentation reactors.

7. The ecological pollution treatment system for livestock and poultry farms based on combination of planting and breeding according to claim 1, wherein the cracking and propagation system is composed of Y cracking and propagation reactors (Y≥2), X cracking reactors (X≥1), Z propagation reactors (Z≥1), steam generators, aeration fans; air filters, electromagnetic valves and connecting pipes; liquid in the adjusting tank is transferred to the cracking and propagation reactors and the cracking reactors by transfer pumps; discharge pipes of the cracking reactors are connected to the propagation reactors; discharge pipes of the cracking and propagation reactors and the propagation reactors are respectively connected to the liquid storage tank through pipes; each cracking and propagation reactor comprises a support, a tank body, a sealing cover and a conveying device; each tank body is fixed on a base; one side of each tank body is sealed by the sealing cover; a cover sealing door is mounted at the other side of each tank body; each cover sealing door is hinged to the tank body; the cover sealing door is locked and sealed with a plurality of locking bolts when, closed, so that a closed cracking and propagation space is formed among the sealing cover, the tank body and the cover sealing door; the tank body is horizontally arranged; guide rails parallel to an axis of the tank body and radially fixed along the tank body are arranged in the tank body for bearing and conveying net cages carrying the sick and dead pigs and the placentas; aeration pipes and a plurality of aeration heads, are mounted at lower parts of the guide rails; one end of each aeration pipe is sealed, while the other end extends to the outside of the tank body by the sealing cover and is sequentially communicated with air outlets of the electromagnetic valve, a one-way valve, the air filters and the aeration fan and steam outlets of the electromagnetic valve, the one-way valve and a steam generator; an exhaust pipe and a feed pipe are arranged at an upper side of each sealing cover; a pressure sensor and a safety valve are further mounted at the upper side of the sealing cover; a temperature sensor and a discharge pipe are mounted at a lower side of the sealing cover; a water jacket is mounted outside each horizontally arranged tank body for cooling each cracking tank; a circulating water inlet pipe of the water jacket is arranged at a lower part of the tank body; a circulating water drainage pipe of the water jacket is arranged at an upper part of the tank body; and the water jacket is covered with the thermal insulation layer made of the thermal insulation material.

8. The ecological pollution treatment system for livestock and poultry farms based on combination of planting and breeding according to claim 1, wherein the heating and heat balancing system is composed of an atmospheric pressure hot water boiler, E high-temperature thermal insulation water tanks (E≥1), F low-temperature thermal insulation water tanks (F≥1), circulating water pumps, electromagnetic valves and connecting pipes; for regions with abundant solar energy resources, the heating and heat balancing system also comprises a solar heating system; the high-temperature water tanks are used to provide water sources for the atmospheric pressure hot water boiler, the solar heating system and the steam generator; water outlet pipes of the high temperature water tanks are respectively communicated with water inlets of the atmospheric pressure hot water boiler, the solar heating system and the steam generator; the water outlets of the atmospheric pressure hot water boiler and the solar heating system are communicated with water inlet pipes of the high-temperature water tanks through the respective pipe; the other water outlet pipe of each high-temperature water tank is connected with the water pump to respectively convey hot water to the high-temperature aerobic solid fermentation reactors, the medium-temperature anaerobic liquid fermentation reactors, the cracking and propagation reactors and the cracking reactors; return water of each reactor is sent back to each high-temperature water tank through respective water returning pipe; the low-temperature water tanks provide water sources for the high-temperature water tanks; the low-temperature water tanks are arranged above the high-temperature water tanks and automatically replenish water to the high-temperature water tanks under control of the detection and control system; the other water outlet pipe of each low-temperature water tank is respectively connected with the water jackets of the cracking and propagation reactors and the cracking reactors by pumps; and water is sent back to the low-temperature water tanks by each water jacket through respective water returning pipes to realize circulation.

9. The ecological pollution treatment system for livestock and poultry farms based on combination of planting and breeding according to claim 1, wherein the waste gas treatment system comprises an odor treatment system of the high-temperature aerobic solid fermentation system and a waste gas treatment system of the cracking and propagation system; the odor treatment system of the high-temperature aerobic solid fermentation system has the following structural connection: the exhaust ports of the high-temperature aerobic solid fermentation reactors, are respectively connected with waste gas inlets of heat exchange condensers through respective exhaust pipe; a waste gas outlet of each heat exchange condenser is respectively connected with an input end of a draught fan through the pipe; air inlets of the heat exchange condenser are communicated with atmosphere; air outlets of the heat exchange condensers are respectively connected with the gas inlets of the high-temperature aerobic solid-fermentation reactors through the pipes; an output end of each draught fan is respectively connected to a gas inlet of a biological deodorization filter tower in parallel through the pipe; an exhaust port of the biological deodorization filter tower is communicated with the atmosphere through a vertical pipe; the waste gas treatment system of the cracking and propagation system has the following structural connection: the aeration ports of the cracking and propagation reactors are respectively connected with the air filters and the aeration fans through the pipes; exhaust pipes of the cracking and propagation reactors and the cracking reactors are respectively connected to input ends of the draught fans; the output ends of the draught fans are connected with gas inlets of the biological deodorization filter towers through the pipes; and the exhaust ports of the biological deodorization filter towers are communicated with the atmosphere through the vertical pipes.

10. An ecological pollution treatment method for livestock and poultry farms based on combination of planting and breeding, comprising the following steps:
I. source separation and water saving of enclosure: rainwater and sewage, drinking water and sewage are separated; the rainwater and the remaining drinking water are discharged to outdoor ditches instead of being mixed into manure; water-saving enclosure flushing and mechanical manure scraping or manual dry manure cleaning is adopted to prevent the rainwater and the remaining drinking water from mixing into the manure at the source of an enclosure and minimize the manure; the enclosure is cleaned with high-pressure spray nozzles or even high-pressure air; the manure cleaned by mechanical manure scraping or manual dry manure cleaning is piled up in a dry manure shed; then, the manure is conveyed to a high-temperature aerobic solid fermentation system and an animal-derived feed factory by the manure shed, respectively; and manure liquid and enclosure flushing water are conveyed to an adjusting tank;
II. feed insect breeding: a water content of the manure is adjusted to an appropriate range at first; the manure is used as an insect feed for breeding insects to obtain insects and ova, and is used as an animal-derived feed, which is mixed with a plant-derived feed at a certain proportion and then is added with appropriate grains, trace elements and other ingredients to produce complete nutrition feed for breeding the livestock and poultry; and frass is conveyed to high-temperature aerobic solid fermentation reactors by the conveying device for high-temperature aerobic fermentation to obtain a solid organic fertilizer;
III. high-temperature aerobic fermentation of manure:
(1) tap water is automatically replenished to low-temperature thermal insulation water tanks; the low-temperature thermal insulation water tanks automatically replenish the water to high-temperature thermal insulation water tanks by utilizing a height difference under control of a detection and control system; circulating pumps of an atmospheric pressure hot water boiler and a solar heating system are started; the water of the high-temperature thermal insulation water tanks is transferred to the atmospheric pressure hot water boiler and the solar heating system by the circulating pumps-for heating and then is transferred to the high-temperature thermal insulation water tanks for, energy storage; electromagnetic valves at front ends of heating water jackets or coils of the high-temperature aerobic solid fermentation reactors are started; hot water circulating pumps are started; and hot water is conveyed to the heating water jackets or the coils of the high-temperature aerobic solid fermentation reactors by the circulating water pumps and the pipes to heat up materials in the high-temperature aerobic solid fermentation reactors;
(2) the manure, the frass and auxiliary materials separated from the enclosure and thermophilic decomposing bacteria are conveyed to the high-temperature aerobic solid fermentation reactors by conveying equipment; the water content of the materials is controlled at 55-65%; and the detection and control system simultaneously starts driving devices of the high-temperature aerobic solid fermentation reactors while adding the materials, so that feeding and stirring are realized in the reactors;
(3) after completing feeding, the detection and control system controls the high-temperature aerobic solid fermentation reactors to stop stirring for a time T1, to stir for a time T2, to stop stirring for the time T1, and then to stir for the time T2; a cycle of stop-stir-stop-stir-stop-stir is a timed stirring program; meanwhile, the detection and control system automatically starts a draught fan to supply oxygen to fermented materials in the high-temperature aerobic solid fermentation reactors when the high-temperature aerobic solid fermentation reactors stir at the time T2; <1> the hot water enters the heating jackets or coils to rise the temperature of the materials in the reactors when the detection and control system detects that the temperature of the materials in the high-temperature aerobic solid fermentation reactors is lower than a set temperature H1 of the materials; <2> the timed stirring program is stopped and changed to a temperature-controlled stirring program when the temperature of the materials in the high-temperature aerobic solid fermentation reactors is greater than or equal to H2; the draught fans are started, and the high-temperature aerobic solid fermentation reactors are driven to stir; the timed stirring program is not started until the temperature of the materials in the high-temperature aerobic solid fermentation reactors is lower than H2; the temperature of the materials in the high-temperature aerobic solid fermentation reactors is maintained in H1-H2; and the timed stirring program and the temperature-controlled stirring program of the high-temperature aerobic solid fermentation reactors are adopted to establish an appropriate fermentation temperature and provide sufficient oxygen for the materials in the high-temperature aerobic solid fermentation reactors and establish an appropriate environment for high-temperature aerobic fermentation of manure solids;
(4) a single fermentation is completed by feeding the materials and fermenting the materials for a tune T3; the detection and control system controls the high-temperature solid fermentation reactors to stop, discharge a part of the materials, then immediately feed the same amount of materials, discharge a part of the materials every T3 time, and then immediately feed the same amount of materials; similarly, a discharge machine is started at first; and meanwhile, the detection and control system controls the high-temperature solid fermentation reactors to stir and guide the discharge;
(5) the materials discharged from the high-temperature aerobic solid fermentation reactor are conveyed to the aging chamber by the conveying device; and the materials are regularly turned or aerated in this period so that the materials are cooled and lose water until the materials are completely decomposed to prepare an organic fertilizer;
(6) the detection and control system respectively detects the temperature of the materials in each high-temperature aerobic solid fermentation reactor when the M high-temperature aerobic solid fermentation reactors simultaneously ferment, so that the temperature of the materials in each high-temperature aerobic solid fermentation reactor is maintained at H1-H2;
(7) the detection and control system detects and controls the water temperature in the high-temperature thermal insulation water tank to keep the water temperature constant in H3-H4; <1> the circulating pump and the atmospheric pressure hot water boiler are started to heat up the hot water in the high-temperature thermal insulation water tank when the temperature in the high-temperature thermal insulation water tank is lower than H3; and the circulating pump of the solar heating system is started to heat up the hot water in the high-temperature thermal insulation water tank when the temperature of the hot water in a heat collecting water tank of the solar heating system is greater than H3; and <2> the atmospheric pressure hot water boiler is shut down when the temperature in the high-temperature thermal insulation water tank reaches H4;
IV. medium-temperature anaerobic fermentation of manure liquid:
(1) a mixture of the manure liquid and the enclosure flushing water is conveyed into the adjusting tank; so that a liquid level of the liquid in the adjusting tank keeps rising; the manure liquid naturally flows into the first soft anaerobic fermentation bag along a connecting pipe due to a height difference when the liquid level is higher than a liquid outlet of the adjusting tank; the detection and control system controls to open the electromagnetic valve at the front end of the heating coil of the first medium-temperature anaerobic liquid fermentation reactor, and the circulating water pump is started, so that the hot water enters the heating coil for internal circulation to rapidly rise the temperature of the materials in the soft anaerobic fermentation bag to a set temperature, and the materials start to perform a medium-temperature anaerobic fermentation reaction;
(2) the liquid naturally flows into the second soft anaerobic fermentation bag along the connecting pipe due to the height difference when the liquid level of the liquid in the first soft anaerobic fermentation bag gradually rises and is higher than the liquid outlet; the detection and control system controls to open the electromagnetic valve at the front end of the heating coil of the second medium-temperature anaerobic liquid fermentation reactor, so that the hot water enters the heating coil for internal circulation to rapidly rise the temperature of the materials in the soft anaerobic fermentation bag to a set temperature, and the materials keep performing the medium-temperature anaerobic fermentation reaction;
(3) the manure liquid naturally flows out along the connecting pipe due to the height difference when the liquid level of the liquid in the second soft anaerobic fermentation bag gradually rises and is higher than the liquid outlet, and so on, the manure liquid directly flows into the liquid outlet tank through the Nth soft anaerobic fermentation bag; the detection and control system controls to open the electromagnetic valve at the front end of the heating coil of the Nth medium-temperature anaerobic liquid fermentation reactor, so that the hot water enters the heating coil for internal circulation to rapidly rise the temperature of the materials in the Nth soft anaerobic fermentation bag to the set temperature, and the materials keep performing the medium-temperature anaerobic fermentation reaction;
(4) polarizers in the N medium-temperature anaerobic liquid fermentation reactors are regularly started, respectively, to prevent the liquid in the soft anaerobic fermentation bags from “crusting” and slow down sedimentation of liquid sediments;
(5) the detection and control system respectively controls on-off of the electromagnetic valve in front of the heating coil of each medium-temperature anaerobic liquid fermentation reactor, and respectively controls the temperature of the materials in each soft anaerobic fermentation bag to keep the temperature constant within a set temperature range; the manure liquid sequentially flows through N soft anaerobic fermentation bags; and fermentation liquid in the Nth soft anaerobic fermentation bag naturally flows into the liquid outlet tank along the pipe due to the height difference, to prepare biogas slurry;
(6) the detection and control system regularly starts the sludge pump to control the liquid level of the liquid outlet tank according to a set anaerobic fermentation time T and ensures that a residence time of the manure liquid in the medium-temperature anaerobic liquid fermentation reactor reaches T; the biogas slurry in the liquid outlet tank is pumped into the liquid storage tank by the sludge pump after the time for anaerobic fermentation reaches T, so that the liquid level of the liquid in the liquid outlet tank is lowered; and the sludge pump is turned off after the detection and control system detects that the liquid level of the liquid outlet tank reaches a lower limit of the liquid level;
(7) the biogas generated. by the N soft anaerobic fermentation bags is conveyed to a biogas pretreatment device through the conveying pipe for treatment, and then is supplied as a combustion fuel to the atmospheric pressure hot water boiler and the steam generator; and the atmospheric pressure hot water boiler and the steam generator also use electricity, diesel, biomass, etc. as supplementary fuels when the temperature is low in winter;
V. cracking and propagation of sick and dead livestock and poultry and placentas:
(1) cracking of sick and dead livestock and poultry and placentas:
<1> a forklift or other transfer equipment is used to place relatively large sick and dead livestock into the net cage; the net cage is pushed into the cracking and propagation reactors by the conveying device; the relatively small sick and dead livestock and placentas are placed into the net cage; the net cage is pushed into the cracking reactors by the conveying device; the cover sealing door is closed; and the manure liquid in the adjusting tank is conveyed into the cracking and propagation reactors and the cracking reactors by the sludge pumps so that the net cages are semi-immersed in the liquid;
<2> the steam generator is started; the electromagnetic valves on steam inlet pipes of the cracking and propagation reactors and the cracking reactors are respectively opened; hot steam generated by the steam generators is respectively conveyed into the cracking and propagation reactors and the cracking reactors by the one-way valves so that the temperature and the pressure of the liquid in the cracking and propagation reactors and the cracking reactors are increased; the exhaust valves on the exhaust pipes of the cracking and propagation reactors and the cracking reactors are respectively closed after exhausting cold air in the cracking and propagation reactors and the cracking reactors, so that the temperature and the pressure in the cracking and propagation reactors and the cracking reactors are continuously increased to respectively reach the temperature and the pressure of statutory treatment; the sick and dead livestock and poultry and the placentas start to be cracked at high temperature and high pressure; and the detection and control system detects and controls the temperature and the pressure in the cracking and propagation reactors and the cracking reactors to keep them constant within a statutory range of the temperature and the pressure for a statutory time, so that the sick and dead livestock and poultry are completely harmless, and are disintegrated and dissolved in the liquid;
<3> the electromagnetic valves on steam inlet pipes of the cracking and propagation reactors and the cracking reactors are respectively closed after high-temperature and high-pressure cracking is completed; the steam generators are turned off after all the reactors complete the high-temperature and high-pressure cracking; the electromagnetic valves on the hot water pipes and the electromagnetic valves on the water inlet pipes of the cracking and propagation. reactors and the cracking reactors are respectively opened; the circulating water pumps on the water outlet pipes of the high-temperature thermal insulation water tanks are started; hot water respectively enters water jackets of the cracking and propagation reactors and the cracking reactors for circulation so that the cracking liquid is cooled and balanced with the temperature of the hot water in the high-temperature thermal insulation water tank; then, the electromagnetic valves on high-temperature hot water pipes are closed; the circulating water pumps on the water outlet pipes of the low-temperature thermal insulation water tanks are started; cold water enters the water jackets of the cracking and propagation reactors and the cracking reactors for circulation so that the cracking liquid is cooled to a set temperature H6; and the circulating water pumps are turned off;
(2) the cracking liquid is used as a culture medium for propagating microorganisms to obtain a microbial culture solution; and the cracking liquid is used as a raw material of the feed insect breeding for breeding feed insects;
(I) a method for propagating the microbial culture solution with the cracking liquid comprises:
<1> the electromagnetic valves on discharge pipes of the cracking reactors are opened; the cracking liquid in the cracking reactors is conveyed into the cracking and propagation reactors; pre-cultured microbial seed liquid is respectively conveyed into the reactors through the feed ports of the cracking and propagation reactors and the propagation reactors; the electromagnetic valves on the aeration pipes of the cracking and propagation reactors and the propagation reactors are respectively opened; the aeration fans are started; fresh air is filtered by the air filters and then is regularly aerated to supply oxygen to the cracking and propagation reactors and the propagation reactors by the one-way valves; meanwhile, the detection and control system detects and controls the temperature in the reactors to keep the temperature in H5-H6; and a detection and control method comprises: the detection and control system controls the high-temperature thermal insulation water tanks to heat the cracking and propagation reactors and the propagation reactors to H6 when the detection and control system detects that the temperature in the cracking and propagation reactors and the propagation reactors is lower than a lower limit H5, and then is turned off;
<2> a propagation process is completed when the concentration of a culture bacteria solution reaches requirements after the cracking liquid is cultured and propagated for a period of time; the microbial culture solution is discharged into the liquid storage tanks through discharge valves for standing, and then is separated by an oil-water separator to obtain the microbial culture solution and grease; and the grease is used as an industrial raw material;
<3> the detection and control system detects and controls the temperature and the pressure of the materials in each reactor according to different cracking and propagation stages when a plurality of reactors react at the same time, so that the temperature and the pressure in each reactor are maintained within the set range;
(II) a method for breeding the feed insects with the cracking liquid comprises:
<1> the livestock and poultry manure is added into the cracking liquid and mixed uniformly; a mixture is used as an insect feed for breeding insects to obtain insects and ova, and is used as an animal-derived feed, which is mixed with a plant-derived feed at a certain proportion and then is added with appropriate grains, trace elements and other ingredients to produce complete nutrition feed for breeding the livestock and poultry; and <2> the residual mixture of the cracking liquid and the livestock and poultry manure as well as frass are conveyed to the high-temperature aerobic solid fermentation reactors by the conveying device for high-temperature aerobic fermentation to obtain a solid organic fertilizer;
VI. planting of feed crops:
(1) the microbial culture solution is sprayed to the aged solid organic fertilizer in a certain proportion, and is stirred uniformly to obtain a bio-organic fertilizer; the microbial culture solution is added into the biogas slurry in a certain proportion to obtain a microbial liquid fertilizer; and an appropriate amount of NPK fertilizer is added into the biogas slurry to prepare an organic-inorganic compound liquid fertilizer according to growth demands of the feed crops;
(2) the appropriate amount of bio-organic fertilizer, microbial liquid fertilizer and organic-inorganic compound liquid fertilizer are respectively applied according to the growth demands of the feed crops before and during planting according to the livestock and poultry breeding quantity of the livestock and poultry farms and the supporting planting land with assimilative capacity of the planted feed crops, to obtain feed ingredients such as pasture, corns, etc.; the feed ingredients are conveyed to the feed factory and are processed to prepare the plant-derived feed, which is mixed with the animal-derived feed in a certain proportion and then is added with appropriate grains, trace elements and other ingredients to produce the complete nutrition feed for breeding the livestock and poultry;
VII. waste gas treatment:
(1) treatment for odor fermented by the high-temperature aerobic solid fermentation system: the electromagnetic valves on the exhaust pipes of the high-temperature aerobic solid fermentation reactors are respectively opened; the odor generated by the high-temperature aerobic solid fermentation reactors during fermentation is respectively introduced into a biological deodorization filter tower by the draught fans after respectively exchanging heat by heat exchange condensers, and is discharged after being absorbed by bio-fillers in the biological deodorization filter tower and transformed to reach a standard; meanwhile, the hot air heated by the heat exchange condensers is respectively introduced into the high-temperature aerobic solid fermentation reactors;
(2) treatment for waste gases fermented by the cracking and propagation system: the electromagnetic valve on the exhaust pipe of each reactor is opened; the waste gases generated by the cracking and propagation system during fermentation are respectively introduced into the biological deodorization filter tower by the draught fans, and are discharged after being absorbed by the bio-fillers in the biological deodorization filter tower and transformed to reach the standard.
